(19) United States
(12) Patent Application Publication
(10) Pub. No.: US 2021/0316110 A1
(43) Pub. Date: Oct. 14, 2021

(54) Medical Treatment Device and Method for Stimulating Neurons of a Patient to Suppress a Pathologically Synchronous Activity of the Neurons

(71) Applicant: Gretap AG, Niederurnen (CH)

(72) Inventor: Peter Alexander Tass, Tegernsee (DE)

(21) Appl. No.: 17/272,452

(22) PCT Filed: Sep. 3, 2019
(86) PCT No.: PCT/EP2019/073493

§ 371 Date: Mar. 1, 2021

Related U.S. Application Data
(60) Provisional application No. 62/726,330, filed on Sep. 3, 2018.

(51) Int. Cl. A61M 21/00; A61N 1/04; A61N 1/36

(52) U.S. Cl. CPC A61M 21/00; A61N 1/0484; A61N 1/36025; A61N 1/36031; A61N 1/36034; A61M 2021/0022; A61M 2205/33; A61M 2021/0055; A61M 2021/0066; A61M 2021/0072; A61M 2021/005; A61M 2230/10; A61M 2205/054; A61M 2021/0027

(57) ABSTRACT

The present invention relates to a medical treatment device for stimulating neurons of a patient. The device comprises a first non-invasive stimulating device for generating at least two different first stimuli to a patient's body, a second non-invasive stimulating device for generating at least two different second stimuli to the patient's body, and a control unit for selectively and intermittently actuating the stimulating devices. In a first operating mode, the control unit is configured to actuate the first stimulating device in a sequence of successive actuating periods such that a number n of first stimuli to be generated simultaneously during the actuating periods is variedly determined across the sequence and to actuate the second stimulating device so as to generate the second stimuli to be paired to the generation of at least a part of the first stimuli. In a second operating mode, the control unit is configured to actuate the second stimulating device so as to generate the second stimuli to be de-coupled from the generation of at least a part of the first stimuli.

### TECHNICAL FIELD

The invention relates to a medical treatment device and a respective medical treatment method for stimulating neurons of a patient to suppress a pathologically synchronous activity of the neurons, i.e. by employing principles of conditioning and/or associative learning.

### TECHNOLOGICAL BACKGROUND

Several brain disorders, such as Parkinson's disease, are characterized by abnormally strong synchronous activity of neurons, i.e. strongly synchronized neuronal firing or bursting. Besides Parkinson's disease, this may also apply, for example, to essential tremor, dystonia, dysfunction after stroke, epilepsy, depression, migraine, tension headache, obsessive-compulsive disorder, irritable bowel syndrome, chronic pain syndromes, pelvic pain, dissociation in borderline personality disorder and post-traumatic stress disorder.

The pharmacological treatment for Parkinson's disease with, for example, L-DOPA may have limited therapeutic effects and it may cause significant long-term side effects. High-frequency Deep Brain Stimulation (DBS) for Parkinson's disease is a standard for medically refractory patients in advanced stages of Parkinson's disease. However, DBS requires surgical procedures associated with a significant risk. For instance, depth electrode implantation in dedicated target areas in the brain may cause bleedings. Furthermore, standard continuous high-frequency DBS may cause side effects.

Further, a non-invasive, vibrotactile multichannel stimulation treatment is known to counteract Parkinsonian signs. The disadvantage of this non-invasive approach lies within an inherently periodic structure of employed stimulations. As to substance, if particular stimulation parameters, such as the repetition rate of sequences of stimuli, are not properly tuned to the dominant frequency of the abnormally active neurons, the stimulation may be ineffective. In particular, in a non-invasive setup, it is difficult to obtain reliable estimates of frequency characteristics of abnormal brain activity due to limitations of chronic non-invasive electroencephalography (EEG) recordings. More importantly, several brain disorders are characterized by abnormal brain rhythms of different frequencies, e.g., around 4 Hz to 5 Hz related to Parkinson's tremor, as opposed to 9 Hz to 35 Hz related to bradykinesia and rigor in Parkinson's disease. Furthermore, multiple central oscillators (i.e. brain rhythms) cause the tremor in different extremities of patients with Parkinson's disease. Without feedback signals from chronically implanted brain electrodes it may be, hence, difficult to achieve optimal stimulation results with the stimulation patterns used so far.

It has been found that abnormally upregulated synaptic connections may lead to abnormal synchronous activity of neurons. However, repeated coincident activation of neurons may lead to an increase of the strength of their mutual synaptic connections. Thus, when the stimulations generated in the known non-invasive vibrotactile multichannel stimulation treatment repeatedly and coincidently overlap with the pathologically synchronous activity of the neurons, the treatment may even cause an unintentional strengthening of the pathologically synchronous activity of the neurons.

Furthermore, for ensuring a therapeutic effect, the known vibrotactile multichannel stimulation treatment requires the patient to receive stimulation for a long period of time, for example, two hours per day during a period of weeks or months. This requires compliance, i.e. willingness of the patient to carry out the therapy, and may be inconvenient, in particular if the stimulation impedes everyday activities. For instance, vibrotactile stimulation delivered to the fingertips of a patient's hand or to both hands may hinder the patients significantly and, hence, reduce compliance. To that end, patients typically do not like a treatment in public via vibratory or electrical stimulators to be applied to the patient's skin, i.e. to the patient's face.

Typically, the known vibrotactile multichannel stimulation treatments are particularly effective if large brain volumes and, hence, large skin areas are stimulated. However, having vibratory and/or electrical stimulators attached to larger skin areas, i.e. to different parts of the body, may be experienced by the patient as even more unpleasant or bothersome. This particularly applies when the treatment is employed for long hours every day during weeks or months.

### SUMMARY OF THE INVENTION

In view of the technical background, it is an object of the present invention to provide an improved non-invasive medical treatment device and a respective medical treatment method which ensure a more convenient treatment for the patient and, at the same time, enable to robustly and effectively suppress pathologically synchronous activities of patient's neurons.

The object is solved by means of a medical treatment device with the technical features of claim 1, a medical treatment device with the technical features of claim 37 and a medical treatment method with the technical features of claim 41.

Accordingly, in a first aspect, a medical treatment device is proposed for stimulating neurons of a patient to suppress a pathologically synchronous activity of the neurons. The device comprises a first non-invasive stimulating device for generating at least two different first stimuli to a patient's body, a second non-invasive stimulating device for generating at least two different second stimuli to the patient's body, and a control unit for selectively and intermittently actuating the first and the second stimulating device. Specifically, the control unit is configured to, in a first operating mode, actuate the first stimulating device in a sequence of successive actuating periods such that a number n of first stimuli to be generated simultaneously during the actuating periods is variedly determined across the sequence and to actuate the second stimulating device so as to generate the second stimuli to be paired to the generation of at least a part of the first stimuli. Further, in a second operating mode, the control unit is configured to actuate the second stimulating device so as to generate the second stimuli to be de-coupled from the generation of at least a part of the first stimuli.

According to a further aspect, a medical treatment device is proposed for stimulating neurons of a patient to suppress a pathologically synchronous activity of the neurons. The device comprises a first non-invasive stimulating device for generating at least two different first stimuli to a patient's body and a second non-invasive stimulating device for generating at least two different second stimuli to the patient's body, wherein each of the first and the second stimuli are configured to, when being administrated to the patient's body, suppress the pathologically synchronous activity of neurons. Further, the device comprises a control unit for selectively and intermittently actuating the first and the second stimulating device. The control unit is configured to operate the first and the second stimulating device in different operating modes. Specifically in a first operating mode, the control unit is configured to actuate the first stimulating device in a first sequence of successive actuating periods, during which the first stimulating device generates at least one first stimuli, and to actuate the second stimulating device so as to generate the second stimuli to be paired to the generation of at least a part of the first stimuli. In a second operating mode, the control unit is configured to actuate the second stimulating device so as to generate the second stimuli to be de-coupled from the generation of at least a part of the first stimuli. Further, in a third operating mode, the control unit is configured to actuate the second stimulating device in a second sequence of successive actuating periods, during which the second stimulating device generates at least one second stimuli, and to actuate the first stimulating device so as to generate the first stimuli to be paired to the generation of at least a part of the second stimuli. Still further, in a fourth operating mode, the control unit is configured to actuate the first stimulating device so as to generate the first stimuli to be de-coupled from the generation of at least a part of the second stimuli.

According to a further aspect, a medical treatment method is proposed for stimulating neurons of the patient to suppress a pathologically synchronous activity of the neurons. The method comprises a step of providing a first non-invasive stimulating device for generating at least two different first stimuli to a patient's body and a second non-invasive stimulating device for generating at least two different second stimuli to the patient's body. In a further step, the first and the second stimulating device are selectively and intermittently actuated successively in a first and a second operating mode. Specifically, in the first operating mode of the first and the second stimulating device, the first stimulating device is actuated in a sequence of successive actuating periods such that a number n of first stimuli to be generated simultaneously during the actuating periods is variedly determined across the sequence and the second stimulating device is actuated so as to generate the second stimuli to be paired to the generation of at least a part of the first stimuli. In the second operating mode, the second stimulating device is actuated so as to generate the second stimuli to be de-coupled from the generation of at least a part of the first stimuli.

### BRIEF DESCRIPTION OF THE DRAWINGS

The present disclosure will be more readily appreciated by reference to the following detailed description when being considered in connection with the accompanying drawings in which:

FIG. 1 is a schematic illustration of a medical treatment device for stimulating neurons of a patient to suppress a pathologically synchronous activity of the neurons.

FIG. 2 schematically shows a first sequence of actuating periods in a first operational mode, according to which a first and a second non-invasive stimulating device of the medical treatment device depicted in FIG. 1 are actuated during the first operational mode.

FIG. 3 schematically shows a second sequence of actuating periods of a second operational mode, according to which the stimulating devices of the medical treatment device are actuated during the second operational mode.

FIG. 4 shows a flow diagram illustrating a procedure employed by a control unit of the medical treatment device for generating the sequence depicted in FIGS. 2 and 3.

FIG. 5 schematically shows a third sequence of actuating periods of a third operational mode, according to which the stimulating devices of the medical treatment device are actuated during the third operational mode.

FIG. 6 schematically shows a fourth sequence of actuating periods of a fourth operational mode, according to which the stimulating devices of the medical treatment device are actuated during the fourth operational mode.

FIGS. 7 to 12 schematically show further embodiments of the medical treatment device.

### DETAILED DESCRIPTION OF PREFERRED EMBODIMENTS

In the following, the invention will be explained in more detail with reference to the accompanying figures. In the Figures, like elements are denoted by identical reference numerals and repeated description thereof may be omitted in order to avoid redundancies.

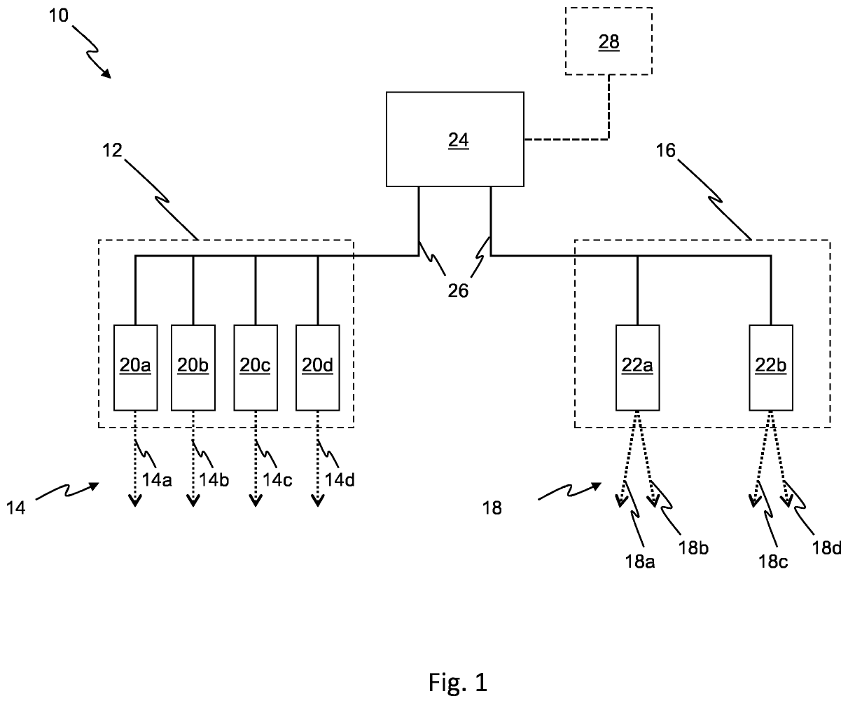

FIG. 1 schematically shows a medical treatment device 10 for stimulating neurons of a patient to suppress a pathologically synchronous activity of the neurons by employing principles of conditioning and/or associative learning.

The medical treatment device 10 is intended to be used for the treatment of neurological or psychiatric diseases, in particular, Parkinsons's disease, essential tremors, dystonia, etc. To that end, the medical treatment device 10 may also be used for the treatment of other neurological or psychiatric diseases, such as epilepsy, tremors as a result of Multiple Sclerosis as well as other pathological tremors, depression, movement disorders, diseases of the cerebellum, obsessive compulsive disorders, Tourette syndrome, functional disorders following a stroke, spastics, tinnitus, sleep disorders, schizophrenia, irritable colon syndrome, addictive disorders, personality disorders, attention deficit disorder, attention deficit hyperactivity syndrome, gaming addiction, neuroses, eating disorders, burnout syndrome, fibromyalgea, migraine, cluster head ache, general head-aches, neuronalgia, ataxy, tic disorder or hypertension, and also for the treatment of other diseases.

The aforementioned diseases can be caused by a disorder of the bioelectric and synaptic communication of groups of neuronal cells which are connected to one another in specific circuits. Hereby, a neuron population generates a continuous pathological neuronal activity and a pathological connectivity (network structure) possibly associated therewith. In this respect, a large number of neurons form synchronous action potentials, this means that the concerned neurons fire or burst excessively synchronously. In addition, the pathological neuron population has an oscillating neuronal activity, this means that the neurons fire or burst rhythmically. In the case of neurological or psychiatric diseases, the mean frequency of the pathological rhythmic activity of the concerned groups of neurons approximately may be in the range of 1 Hz to 60 Hz, but may, however, also be outside of this range. By contrast, the neurons of healthy people fire or burst qualitatively differently, for example, in an uncorrelated manner.

In other words, each of the aforementioned diseases may be characterized by at least one neuronal population in the brain or spinal cord of the patient which has a pathological synchronous neuronal activity. For suppressing such a pathologically synchronous activity, the medical treatment device 10 is configured to stimulate the affected neuronal population so as to cause the affected neural population to fire or burst in an uncorrelated manner, i.e. non-synchronously.

Generally, the proposed medical treatment device 10 is a non-invasive treatment device. This means that the medical treatment device 10 deploys a non-invasive procedure to achieve the intended therapeutic effect. In other words, in an operational state, the medical treatment device 10 is present outside of the patient's body. In other words, the medical treatment device 10 is not implanted into the body, i.e. associated with an intervention procedure into the patient's body.

For acting on the patient's body and thereby stimulating the neurons of the patient, the medical treatment device 10 comprises a first non-invasive stimulating device 12 for generating a plurality of different first stimuli 14 to the patient's body and a second non-invasive stimulating device 16 for generating a plurality of different second stimuli 18 to the patient's body. The considered first and second stimuli 14, 18 generated by the first and the second stimulating device 12, 16 differ from one another.

In general, the stimuli 14, 18 generated by the first and second stimulating device 12, 16 may refer to any excitations capable of being sensed by the patient's body, i.e. by respective receptors. In other words, these stimuli may be sensed by receptors, for example, in the eyes, the ears and/or the skin of the patient depending on the respective stimuli characteristic and are guided from there to a patient's nerve system causing an actuation of neurons in the patient's brain or spinal cord.

Typically, the stimuli characteristic is specified by a stimulation modality and/or a stimuli strength and/or a stimuli frequency and/or a stimuli time course and/or a location of origin and/or a stimulating unit of origin. As regards the stimulation modality, the stimuli may have the stimulation modality of, for example, mechanical stimuli, such as tactile stimuli and/or vibratory stimuli and/or vibrotactile stimuli, and/or electrical stimuli and/or optical stimuli and/or acoustic stimuli and/or chemical stimuli and/or thermal stimuli. The first stimuli 14 generated by the first stimulating device 12 and the second stimuli 18 generated by the second stimulating device 16 may comprise at least one of the aforementioned stimulation modalities or any other suitable stimulation modality.

The first stimuli 14 generated by the first stimulating device 12 are configured to, when being administrated to the patient's body, suppress the pathologically synchronous activity of neurons, in particular in at least one of a patient's brain or spinal cord.

Generally, a suppression of the synchronous activity of neurons may mean that the rate of coincidence activity of the patient's neurons is reduced or that a volume or population of neurons is desynchronized. Reduction of the rate of coincidence activity of the neurons brought about by the stimulation may lead to a reduction of the synaptic weights and thus to an unlearning of the tendency to produce to pathological synchronous activity.

Specifically, for suppressing the neuronal population affected by the pathologically synchronous activity, the first stimulating device 12 is configured to generate the first stimuli 14 to the patient's body which, upon being sensed by receptors of the patient's body and thereafter guided to its nerve system, at least partially cause an actuation of the affected neuronal population. Specifically, the first stimuli 14 are generated and applied to the patient's body so as to cause the affected neural population to fire or burst in an uncorrelated manner, i.e. non-synchronously. For doing so, characteristics of the generated stimuli and an actuation time pattern, according to which the stimuli are to be applied to the patient's body, are respectively set as described in the following in more detail.

Since the first stimuli 14 are therapeutically effective sensoric stimuli, there are also referred to as “specific” stimuli. In the present disclosure, specific stimuli refer to a stimulation which causes a long-lasting de-synchronization of abnormal neuronal synchrony if administrated during a sufficiently long time without applying conditioning and/or associative learning approaches or procedures.

As set forth above, it has been found that multichannel stimulation treatments, i.e. using vibrotactile stimuli, are particularly effective if large brain or spinal cord volumes are stimulated. Accordingly, in the proposed medical treatment device 10, this is enabled by providing the first stimulating device 12 which generates mechanical and/or electrical stimuli. As to substance, by generating mechanical and/or electrical stimuli, the first stimulating device 12 enables to stimulate a large skin area of the patient, thereby causing an actuation of large volume of neurons in the patient's brain or spinal cord. In this way, the stimulating treatment provided by the medical treatment device 10 may be performed in an effective way. Accordingly, the first stimuli 14 generated by the first stimulating device 12 comprises or consists of at least one mechanical stimulus, such as tactile stimulus and/or vibratory stimulus and/or vibrotactile stimulus, and/or at least one electrical stimulus.

The first stimulating device 12 is configured to generate or induce the first stimuli 14 to different sides of the patient's body. The first stimulating device 12 comprises a plurality of first stimulating units 20, each of which generates at least one of the first stimuli 14. More specifically, as depicted in FIG. 1, the first generating device 12 comprises four first stimulating units 20a-d, each of which generates first stimuli 14a-d, as illustrated by dashed lines in FIG. 1.

Although the shown embodiment comprises four first stimulating units 20, satisfying therapeutic effects may also be achieved with a medical treatment device having less or more than four first stimulating units 20. Further, in an alternative embodiment, each of the first stimulating units 20 may also be configured to generate more than one first stimulus 14. For example, one of the first stimuli units 20 may be configured to generate four different first stimuli 14, wherein another one of the first stimulating units 20 may be configured to generate more or less than four different first stimuli 14.

The first stimulating units 20 are configured to being fastened to different sites or parts of the patient's body so as to cover a relative large skin area of the patient, thereby providing an effective therapeutic treatment. In other words, the first stimulating units 20 are configured to induce first stimuli 14 to the patient's body when being in contact with a body surface of the patient. Thus, the medical treatment device 10 further comprises fastening means (not shown) for releasably fastening the first stimulating units 20 to the different parts of the patient's body.

Specifically, the first stimulating device 12 with the plurality of first stimulating units 20 may comprise or be provided in the form of at least one medical treatment glove, at least one medical treatment seat pad, at least one medical treatment sole, at least one medical treatment belly band, at least one medical treatment neck band, at least one medical treatment shoulder band, at least one medical treatment voice box and/or at least one medical treatment face mask.

Compared to the first stimuli 14 generated by the first stimulating device 12, the second stimuli 18 generated by the second stimulating device 16 may be, but is not required per se, to be configured to, when being administrated to the patient's body without applying conditioning and/or associative learning approaches or procedures, suppress the pathologically synchronous activity of the affected neurons. Thus, in one embodiment of the medical treatment device 10, the second stimuli 18 may be configured to, when being administrated to the patient's body without applying conditioning and/or associative learning approaches or procedures, suppress the pathologically synchronous activity of neurons, in particular in at least one of a patient's brain or spinal cord. In this case, the second stimuli 18 applied by the second stimulating device 16 may also constitute “specific” stimuli.

Alternatively or additionally, the second stimuli 18 may be configured such that, when being administered to the patient's body without applying conditioning and/or associative learning approaches or procedures, do not necessarily cause a decrease of the amount of abnormal neuronal synchrony in the patient's brain and/or spinal cord. In other words, the second stimuli 18, when taken on their own, i.e. without the cooperation with the first stimuli 14 in the learning phase described in the following, have no or only a small desynchronizing effect on the pathological synchronous neuronal activity of the patient's neurons. In this case, the second stimuli 18 applied by the second stimulating device 16 may also be referred to as “non-specific” stimuli. In other words, the non-specific stimuli do not or only hardly induce a de-synchronization if delivered to the patient's body without applying conditioning and/or associative learning approaches or procedures.

As set forth above, the second stimuli 18 generated by the second stimulating device 16 may comprise at least one of the aforementioned stimulation modalities or any other stimulation modality. For example, the second stimulating device 16 may be configured to generate the second stimuli 18 which are of the same or different stimulation modality as the first stimuli 14. Thus, the second stimulating device 16 may be configured to generate the second stimuli 18 comprising at least one of mechanical stimulus and/or electrical stimulus. Alternatively or additionally, the second stimuli 18 may comprise optical stimuli and/or acoustic stimuli and/or chemical stimuli and/or thermal stimuli.

For example, the second stimulating device 16 may comprise or be provided in the form of at least one medical treatment visual stimulation unit for generating visual or optical stimuli and/or a medical treatment acoustic stimulator for generating acoustic stimuli. Alternatively or additionally, the second stimulating device 16 may be configured to provide mechanical stimuli and/or electrical stimuli and may comprise or be provided in the form of at least one medical treatment glove, at least one medical treatment seat pad, at least one medical treatment sole, at least one medical treatment belly band, at least one medical treatment neck band, at least one medical treatment shoulder band and/or at least one medical treatment face mask.

The second stimulating device 16 may comprise one or more second stimulating units 22, each of which generates at least one of the second stimuli 18. In the shown embodiment of the medical treatment device 10, the second stimulating device 16 comprises two stimulating units 22a,b in the form of acoustic stimulators, i.e. loudspeakers, each of which generates at least two second stimuli 18a-d as illustrated by dashed lines in FIG. 1.

The second stimulating units 22 are configured to be fastened to the ears of the patient. For that reason, the medical treatment device 10 further comprises suitable fastening means (not shown) for releasably fastening the second stimulating units 22a,b to the patient's head, i.e. ears. Thus, the second stimulating device 16 is configured to generate second stimuli 18 to different sites of the patient's body, in particular to both ears of the patient. More specifically, the first and the second stimuli 14, 18 may be delivered to different body parts and/or with different stimulation modalities.

Although the shown embodiment comprises two stimulating units 22, satisfying therapeutic effects may also be achieved with a medical treatment device 10 having less or more than two stimulating units 22, wherein each of the stimulating units 22 may be configured to generate more or less than two different second stimuli 18. Again, the present invention is not limited to second stimuli 18 in the form of acoustic stimuli. Thus, satisfying therapeutic effects may also be achieved when providing second stimuli 18 having other stimulation modalities.

As set forth above, the considered first and second stimuli 14, 18 generated by the first and the second stimulating device 12, 16 are all different. This means that the first and the second stimuli 14, 18 differ from one another in terms of their stimulation modality and/or their stimuli strength and/or their stimuli frequency and/or their stimuli time course and/or their location of origin and/or their stimulating unit of origin. In the context of the present disclosure, the term “location of origin” may refer to a location at which the respective stimuli is generated or applied to the patient's body and the term “stimulating unit of origin” may refer to the stimulating unit which generates the respective stimuli.

The medical treatment device 10 further comprises a control unit 24 for selectively and intermittently actuating the first and the second stimulating device 12, 16, i.e. the first stimulating units 20 and the second stimulating units 22. The control unit 24 is connected to each one of the first and the second stimulating device 12, 16 via connecting lines 26, through which control signals are guided from the control unit 24 to the first and the second stimulating units 20, 22, respectively, for actuating the same. The connecting lines 26 may be realized wirelessly or by means of connecting wires.

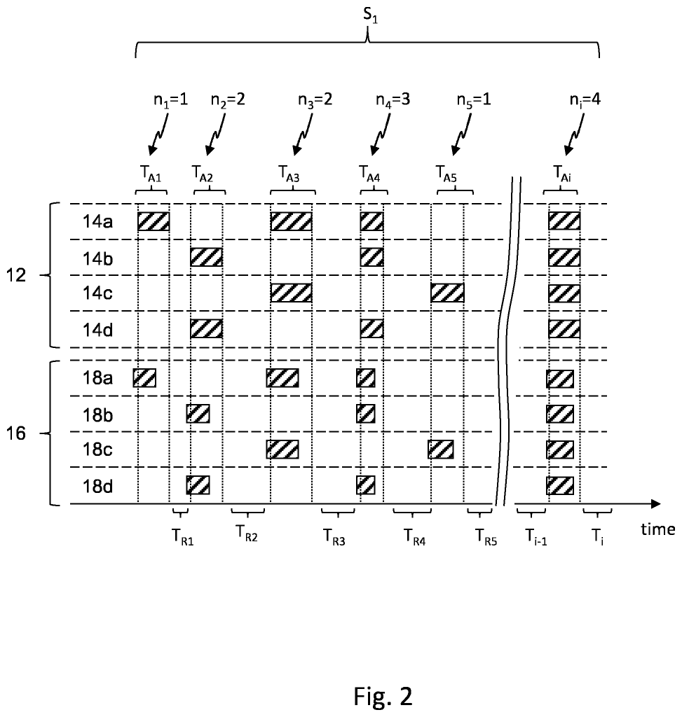

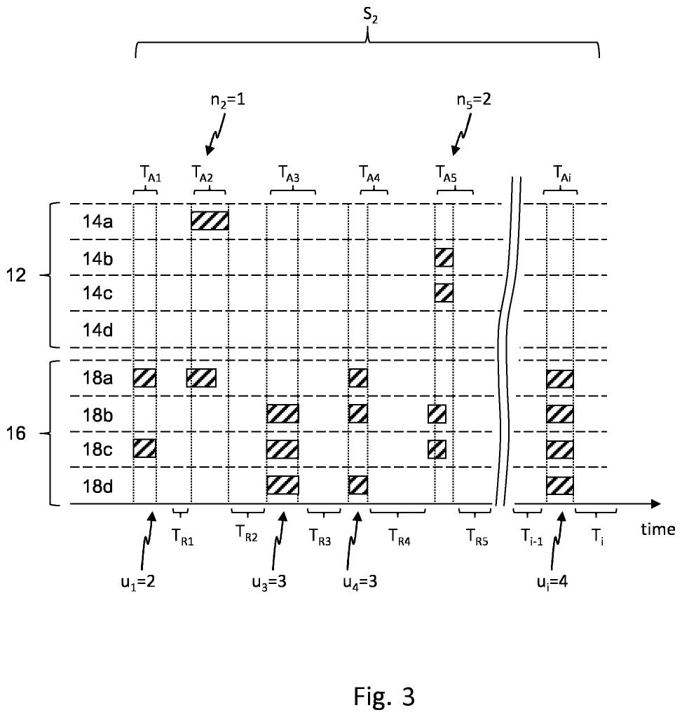

Specifically, the control unit 24 is configured to operate the first and the second stimulating device 12, 16 in a first operating mode, as depicted in FIG. 2, and a second operating mode, as depicted in FIG. 3. Specifically, the control unit 24 is configured to operate, i.e. repeatedly operate, the first and the second stimulating device 12, 16 in the first operating mode and subsequent in the second operating mode.

In the first operating mode, the control unit 24 is configured to actuate the first and the second stimulating device 12, 16 in a first sequence S₁ of successive actuating periods T_{A1-Ai} so as to generate the first stimuli 14a-d and the second stimuli 18a-d, as depicted in FIG. 2, wherein character “i” refers to a total number of actuating periods within the first sequence S₁. More specifically, in the first operating mode, the control unit 24 is configured to actuate the second stimulating device 16 so as to generate the second stimuli 18 to be paired to the generation of at least a part of the first stimuli 14. Specifically, in this context, the term “paired” means that, during the first operating mode, the second stimuli 18 are at least partly administrated to the patient's body coupled in time to the generation of the first stimuli 14. In other words, during the first operational mode, the first and the second stimuli 14, 18 are at least partly administrated in pairs to the patient's body.

Administrating paired first and second stimuli 14, 18 to the patient's body may have the effect that the nerve system of the patient is conditioned and/or associatively learns to react to the second stimuli 18, which may be non-specific stimuli, in the same way or in a slightly attenuated form as to the specific first stimuli 14. Accordingly, the first operating mode may also be referred to as a “learning mode” or “learning phase”. In this mode, a pairing of the first and the second stimuli 14, 18 is performed such that, thereafter, administration of specific or non-specific second stimuli 18 alone provide a de-synchronizing effect that is greater than without pairing.

Again, as the conditioning or associative learning takes place during the first operating mode, the nerve system of the patient may react to the second stimuli 18 in the same way or in a slightly attenuated form as to the specific first stimuli 14 even when the first stimuli 14 are no longer administered to the patient's body. In other words, after the learning phase, the nerve system reacts to the administration of isolated second stimuli 18, i.e. unpaired from the first stimuli 14, as if the specific first stimuli 14 would be applied to the patient's body.

The proposed medical treatment device 10 may take advantage of this effect during the second operating mode. As to substance, in the second operating mode, the control unit 24 is configured to actuate the second stimulating device 16 so as to generate the second stimuli 18 to be de-coupled from the generation of at least a part of the first stimuli 14. Due to the conditioning or associative learning of the patient's nerve system during the first operating mode, the nerve system of the patient reacts to the administration of the de-coupled second stimuli 18 during the second operating mode as if the specific first stimuli 14 would be applied to the patient's body. Thus, the second stimuli 18, which may be non-specific stimuli, when being administrated to the patient's body on their own during the second operation mode, is configured to suppress the pathologically synchronous activity of neurons in the same way or in a slightly attenuated form as the specific first stimuli 14. The second operating mode may also be referred to as the “actual stimulating mode or phase”,

In the second operating mode, as set forth above, the first and the second operating device 12, 16 are actuated such that the generation of the second stimuli 18 takes place isolated, i.e. de-coupled from or without the generation of at least a part of the first stimuli 14. Additionally, the first and the second stimuli 14, 18 partly may also be administered in pairs during the second operating mode in a similar way as compared to the first operating mode. In other words, during some time periods in the second operating mode, the second stimuli 18 may be administrated on their own and, during some other time periods in the second operating mode, may be administrated paired with first stimuli 14. However, as the second stimuli 18 also provide therapeutic effects when being applied on their own due to the conditioning and/or associate learning in the first operating mode, the demand for specific first stimuli 14 and thus for administering paired first and second stimuli 14, 18 is reduced in the second operating mode.

As a result, for performing the intended therapeutic treatment with the medical treatment device 10, the proposed device enables that, for a longer period of time during the therapeutic treatment, the second stimulating device 16 may be actuated alone, i.e. without the first stimulating device 12, such that the patient is only subjected to the second stimuli 18, which may be non-specific stimuli. In this way, by employing the above described conditioning and/or associative learning procedure, instead of stimulating large skin surface areas by actuating the first stimulating device 12, the proposed medical treatment device 10 enables to significantly reduce the integral skin surface area to be stimulated in the course of the treatment, i.e. by significantly reducing the actuating time of the first stimulating device 12. In this way, the therapeutic treatment provided by the medical treatment device 10 may be perceived as more comfortable or less distracting by the patient. Due to the increased comfort on carrying out the therapy, the patient's compliance may be increased and thus the therapeutic result may be improved as a whole.

This particularly applies when the second stimulating device 16 is provided so as to generate second stimuli 18 which, compared to the first stimuli 14 generated by the first stimulating device 12, are perceived as more comfortable by patients. For example, compared to mechanical or electric stimuli generated by the first stimulating device 12, acoustic or optical stimuli generated by the acoustic stimulator or optical stimulator may be perceived as significantly more comfortable by patients.

In the first operating mode, the control unit 24 may be configured to actuate the first and the second stimulating device 12, 16 so as to generate not only pairs of first and second stimuli 14, 18, but also the second stimuli 18 on their own, i.e. isolated or de-coupled from first stimuli 14. In other words, during a part of the actuating periods of the first sequence S₁, the second stimuli 18 may be paired to the generation of the first stimuli 14 and, during the other part of the actuating periods of the first sequence S₁, the second stimuli 18 are generated on their own, i.e. isolated or de-coupled from the first stimuli 14. Alternatively, during the first operating mode, the first and the second stimuli 14, 18 may only be generated in pairs throughout the first sequence S₁.

For enabling that the conditioning and/or the associative learning is performed effectively, the control unit 24 is configured such that, in the first operating mode, a majority of the second stimuli 18, in particular more than 50%, and/or a majority of actuating periods T_{A1-Ai} within the first sequence S₁, in particular more than 50%, is coupled to the generation of at least one first stimuli 14. For example, the control unit 24 may be configured such that, in the first operating mode, substantially 60% or more than 60%, e.g. 60%, 70%, 80%, 90% or 100%, of the second stimuli 18 is coupled to the generation of at least one first stimulus 14. Further, the control unit 24 may be configured such that, in the first operating mode, a majority of the first stimuli 14, in particular more than 50%, and/or a majority of actuating periods T_{A1-Ai} within the first sequence S₁, in particular more than 50%, is coupled to the generation of at least one second stimuli 18. For example, the control unit 24 may be configured such that, in the first operating mode, substantially 60% or more than 60%, e.g. 60%, 70%, 80%, 90% or 100%, of the first stimuli 14 is coupled to the generation of at least one second stimuli 18.

As can be gathered from FIG. 3, in the second operating mode, the control unit 24 is configured to intermittently actuate the first and the second stimulating device 12, 16 in a second sequence S₂ of successive actuating periods T_{A1-Ai} so as to generate the first and the second stimuli 14a-d, 18a-d wherein character “i” refers to a total number of actuating periods within the second sequence S₂. Specifically, in the second operating mode, the first and the second stimulating device 12, 16 are actuated such that, for at least a part of the actuating periods T_{A1-Ai}, the second stimuli 18 are generated on their own, i.e. unpaired or de-coupled from the generation of the first stimuli 14, as depicted in FIG. 3 by actuating periods T_{A1}, T_{A3}, T_{A4}, and T_{Ai}. Further, the first and the second stimulating device 12, 16 are actuated such that, for at least a part of the actuating periods T_{A1-Ai}, the first and the second stimuli 14, 18 are generated in pairs, as depicted in FIG. 3 by actuating period T_{A2} and T_{A5}.

Specifically, for increasing the comfort for a patient during the medical treatment, the control unit 24 is configured such that, in the second operating mode, a majority of the second stimuli 18, in particular more than 50%, and/or a majority of actuating periods within the second sequence S₂, in particular more than 50%, is de-coupled from the generation of the first stimuli 14. For example, the control unit 24 may be configured such that, in the second operating mode, substantially 60% or more than 60%, e.g. 60%, 70%, 80%, 90% or 100%, of the second stimuli 18 is de-coupled from the generation of the first stimuli 14.

To summarize, in the first and the second operating mode depicted in FIGS. 2 and 3, either second stimuli 18 are generated alone, i.e. de-coupled from or without the first stimuli 14, or in pairs with the first stimuli 14. For the better handling, stimuli composed of first and second stimuli 14, 18 are referred to as paired stimuli “P” and stimuli composed of only second stimuli 18 are referred to as isolated stimuli “I”. The controller 24 may be configured to decide for each one of the respective actuating periods T_{A} of the respective Sequence S₁, S₂ whether the second stimuli 18 are generated alone, i.e. “I” stimuli, or in pairs with a first stimulus 14, i.e. “P” stimuli. In other words, the control unit 24 may set a sequential arrangement of “I” and “P” stimuli throughout the sequences S₁, S₂. For example, the control unit 24 may set a periodic sequential arrangement, e.g. for the first operating mode in the following sequence: P-P-P-P-I-I-P-P-P-P-I-I. However, for providing a more robust and more effective treatment, the timely pattern or sequential arrangement of “I” and “P” stimuli may also be selected deterministically or stochastically or combined stochastically-deterministically, e.g. for the second operating mode in the following sequence: P-I-I-I-P-P-P-I-I-I-I-P-P-I-I-I-P-I-I-P-P-I-I-I-I-I-P-I . . . .

Further, for setting an operating mode of the first and the second stimulating device 12, 16, the control unit 24 may be configured to change, in particular successively change, a rate of “I” stimuli, i.e. second stimuli 18 generated de-coupled from the first stimuli 14. For example, the control unit 24 may be configured to switch the medical treatment device 10 from the first operating mode into the second operating mode by increasing, in particular successively increasing, the rate of second stimuli 18 to be generated de-coupled from the generation of the first stimuli 14 relative to a rate of second stimuli 18 to be generated in pairs with the first stimuli 14.

In a further development, the medical treatment device 10 may be configured to output a signal, i.e. a warning signal to a patient, indicative of a change of operating mode of the medical treatment device 10. For example, the medical treatment device 10 may be configured to output a signal indicating that the control unit 24 switches the operating mode from the first operating mode into the second operating mode, and vice versa. In other words, by outputting the signal, the medical treatment device 10 may indicate to the patient that the control unit 24 switches the operating mode of the medical treatment device 10, i.e. of the first and the second stimulating device 12,16, from its first operating mode to its second operating mode, and vice versa. To that end, when the control unit 24 is configured to actuate merely the second stimulating device 16 during the second operating mode, i.e. without actuating the first stimulating device 12, the thus output signal may indicate to the patient that the first stimulating device 12 should be attached to its body when the signal indicates that the medical treatment device 10 is to be operated in the first operating mode. Accordingly, the output signal may indicate to the patient that the first stimulating device 12 is to be detached from its body when the signal indicates that the medical treatment device 10 is to be operated in the second operating mode, during which the first stimulating device 12 is not operated. In this way, the patient may be instructed to attach and/or detach the first stimulating device 12 to or from its body, thereby enabling that the medical treatment, at least during the second operating periods, is perceived as more comfortable or less distracting by the patient. The output signal may be a visual signal or an acoustic signal perceptible to a patient. Alternatively or additionally, the medical treatment device 10 may be configured to transfer the output signal to an external device, such as a mobile device, i.e. a smartphone, which, upon receiving the signal output from the medical treatment device 10, generates an acoustic signal, i.e. via a loudspeaker, and/or a visual signal, i.e. via a display of the mobile device for instructing the patient.

The duration of the first stimuli 14 may be between 30 ms and 250 ms, in particular at around 150 ms, but can, however, also lie outside of this range. The duration of the second stimuli 18 may be between 30 ms and 250 ms, in particular at around 150 ms, but can, however, also lie outside of this range.

When the first and the second stimuli 14, 18 are administrated in pairs, the first stimuli and the second stimuli 14, 18 may overlap. In other words, within the actuating periods T_{A1-Ai}, the first and the second stimuli 14, 18 may overlap. During the period of the overlap, the first and the second stimuli 14, 18 are generated simultaneously. This overlap may amount to e.g. at least 10% or 20% or 30% or 40% or 50% or 60% or 70% or 80% or at least 90% or even 100% of the duration of the first or second stimuli 14, 18.

In the actuating pattern depicted in FIGS. 2 and 3, when paired stimuli are generated in the first and the second operating mode, the generation of the second stimuli 18 is coupled in time to the generation of the first stimuli 14 such that the second stimuli 18 partially overlaps the first stimuli 14 by 10%, but may also overlap by more than 10% as set forth above, in particular by 10% to 100%. Further, as can be gathered from FIGS. 2 and 3, when the first and the second stimuli 14, 18 are generated in pairs in the first and the second operating mode, the second stimuli 18 precedes the first stimuli 14. Alternatively or additionally, when the first and the second stimuli 14, 18 are generated in pairs, also the first stimuli 14 may precede the second stimuli 18.

In the medical treatment device 10, the control unit 24 is configured to generate the first and the second sequence S₁, S₂ of actuating periods T_{A1-Ai}. In the following, the generation of these sequences S₁, S₂ of actuating periods T_{A1-Ai} is described in more detail. It will be obvious to the skilled person that the present invention is not limited to the approach specified in the following. Rather, he will understand that other approaches for generating the sequences may be used without leaving the scope of the present invention.

As set forth above, the control unit 24 is configured to actuate the first and the second stimulating device 12, 16 according to a first sequence S₁ of successive actuating periods T_{A1-Ai} during the first operating mode and according to a second sequence S₂ of successive actuating periods T_{A1-Ai} during the second operating mode, wherein character “i” refers to a total number of actuating periods within the respective sequence S₁, S₂. Specifically, these sequences S₁, S₂ differ from one another by the rate of generated single, isolated second stimuli 18 compared to the rate of paired first and second stimuli 14. Specifically, in the first sequence S₁, the rate of generated paired stimuli is more than 50%, in particular more than 60%, among the actuating periods. By contrast, in the second sequence S₂ the rate of generated paired stimuli is less than 50%, in particular less than 40%, among the actuating periods T_{A1-Ai}.

Each of the first and the second sequence S₁, S₂ form a control sequence or control pattern illustrating the actuation of the stimulating devices 12, 16 over the course of time. Thus, the sequences S₁, S₂ illustrate a time period in which the stimulating devices 12, 16, i.e. stimulating units 20, 22 of the medical treatment device 10 are selectively and intermittently actuated. The first and the second sequence S₁, S₂ of actuating periods T_{A1-Ai} may have a total duration corresponding to a duration of a treatment procedure, e.g. a daily treatment procedure, performed by the medical treatment device 10. During a daily treatment procedure, the medical treatment device may be switched between the first and the second operating mode several times. Thus, the first and/or the second sequence S₁, S₂ may have a total duration between 10 min to 8 hours, e.g. about 120 minutes.

Each of the first and the second sequence S₁, S₂ comprises a number i of time shifted, non-overlapping actuating periods T_{A1-Ai}. In this context, the term “actuating period” refers to a time period, during which the stimuli 14, 18 can be generated and thus can applied to the patient's body. For example, the actuating periods T_{A1-Ai} may have a duration between 30 ms and 250 ms, in particular at around 150 ms. In an alternative embodiment, the actuating periods within the first or second sequence S₁, S₂ may at least partially overlap.

As depicted in FIGS. 2 and 3, between successive actuating periods T_{A1-Ai}, resting periods T_{R1-Ri} are scheduled. In this context, the term “resting period” refers to a time period during which none of the first and the second stimulating device 12, 16 is actuated. Accordingly, during the resting periods T_{R1-Ri}, the patient's body is not subjected to stimuli generated by the first and the second stimulating device 12, 16.

In FIGS. 2 and 3, the generation of the respective stimuli 14, 18 and thus the actuation of the respective stimulating devices 12, 16 are illustrated by dashed fields positioned within the actuating periods T_{A1-Ai}.

In the first sequence S₁ shown in FIG. 2, the control unit 24 actuates the first and the second stimulating device 12, 16 such that, during each one of the actuating periods T_{A1-Ai}, at least one first stimulus 14a-d is generated which is paired or coupled to at least one second stimulus 18. In other words, among the actuating periods T_{A1-Ai} of the first sequence S₁ the control unit 24 is configured to generate paired first and second stimuli, but none isolated first or second stimuli. Although not shown in FIG. 2, the control unit 24 may also be configured to, for at least one or a part of the actuating periods T_{A1-Ai} of the first sequence S₁, generate isolated, i.e. de-coupled second stimuli 18 or first stimuli 14.

In the second sequence S₂ shown in FIG. 3 by contrast, the control unit is configured to actuate the first and the second stimulating device 12, 16 such that during the actuating periods T_{A2} and T_{A5}, at least one first stimulus 14a-d is generated which is paired or coupled to at least one second stimulus 18, and during the actuating periods T_{A1}, T_{A3}, T_{A4} and T_{Ai}, generate isolated, i.e. de-coupled second stimuli 18.

Further, when actuating the first stimulating device 12, the control unit 24 may be configured to exclusively generate one single first stimulus 14 during an actuating period T_{A1-Ai} or, alternatively, generate at least two first stimuli 14 simultaneously during an actuating period T_{A1-Ai}. In the context of the present disclosure, the number of first or second stimuli 14, 18 to be actuated, i.e. simultaneously actuated during the respective actuating periods T_{A1-Ai} is denoted as “n₁₋ᵢ”, wherein n is an integer equal to or greater than one.

In the proposed medical treatment device, the control unit 24, across the first sequence S₁ of actuating periods T_{A1-Ai}, is configured to variedly determine for each actuating period T_{A1-Ai} the number n₁₋ᵢ of first stimuli 14 to be actuated during the respective actuating period T_{A1-Ai}. In this context, the term “variedly” means that the values for the variable n₁₋ᵢ diversely, i.e. non-periodically, varies across the first sequence S₁. In this way, regularities or periodicities actuating pattern may be avoided, thereby contributing to a robust and effective suppression of pathologically synchronous activity of the patient's neurons.

Accordingly, when actuating the second stimulating device 16, the control unit 24 may be configured to exclusively generate one single second stimulus 18 during an actuating period T_{A1-Ai} or, alternatively, generate at least two second stimuli 18 simultaneously during an actuating period T_{A1-Ai}. In this context, the number of first or second stimuli 14, 18 to be actuated, i.e. simultaneously actuated during the respective actuating periods T_{A1-Ai} is denoted as “u₁₋ᵢ”, wherein u is an integer equal to or greater than one.

In the proposed medical treatment device, the control unit 24, across the second sequence S₂ of actuating periods T_{A1-Ai}, is configured to variedly determine for each actuating period T_{A1-Ai} the number u₁₋ᵢ of first stimuli 14 to be actuated during the respective actuating period T_{A1-Ai}. In this context, the term “variedly” means that the values for the variable u₁₋ᵢ diversely, i.e. non-periodically, varies across the second sequence S₂. In this way, regularities or periodicities actuating pattern may be avoided, thereby contributing to a robust and effective suppression of pathologically synchronous activity of the patient's neurons.

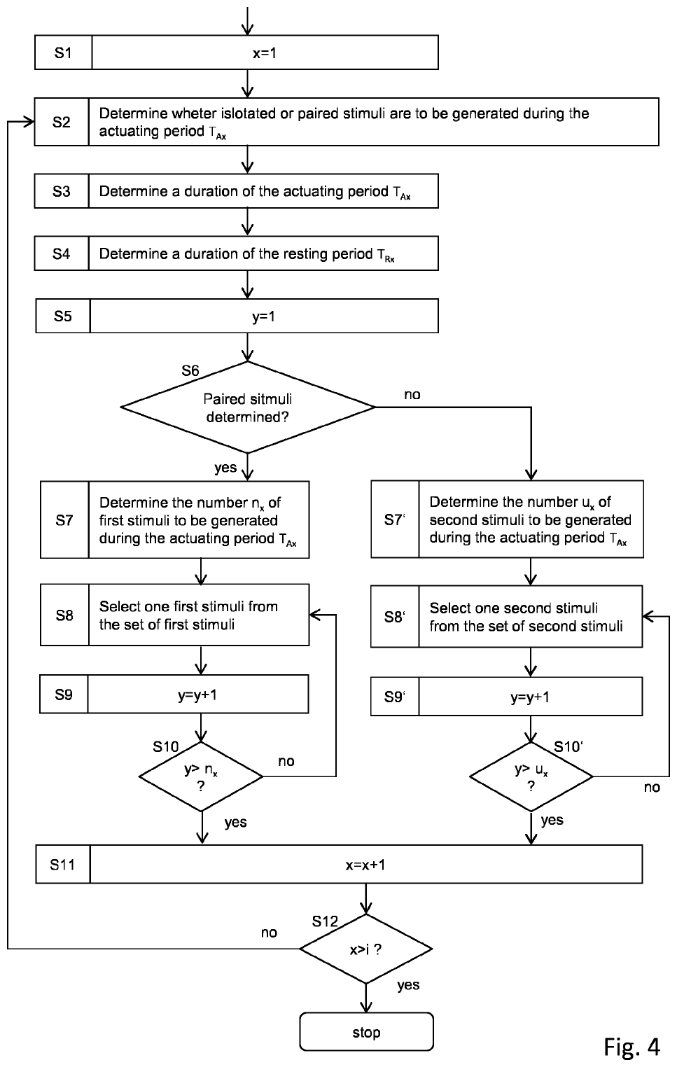

In the following, under reference to FIG. 4 which shows a flow diagram illustrating a procedure employed by the control unit 24, the process of generating a sequence S of actuating periods is specified. The generated sequence may either be the first or the second sequence S₁, S₂ as described above. The procedure may be performed by the control unit 24 prior to actuating the stimulating devices 12, 16 according to the generated sequence S. Alternatively, the procedure may be performed successively during the sequences S, i.e. during actuating the stimulating devices 12, 16.

The sequence S comprises the number i of different actuating periods T_{A1-Ai}, as depicted in FIGS. 2 and 3. In the shown procedure, steps S2 to S12 are repeatedly and successively executed for each one of the actuating periods T_{A1-Ai} within a sequence S.

In a first step S1, a value of a control variable x is set to equal 1. In this way, in the steps S2 to S10 or S10′ of the procedure, initially, the first actuating period T_{A1} of the sequence S is generated.

For each one of the actuating periods T_{Ax}, the control unit 24 determines in step S2, whether at least one isolated second stimulus or a paired first and second stimuli is/are to be generated during the respective actuating period T_{Ax}. Specifically, the control unit 24 may be configured to variedly determine, i.e. stochastically and/or deterministically and/or combined stochastically-deterministically determine, across the actuating periods T_{A1-Ai} whether isolated or paired stimuli are to be generated. For example, for doing so, the control unit 24 may employ an exponential distribution process and/or a Markov process and/or any other suitable stochastic or deterministic or combined stochastic-deterministic process.

As set forth above, a rate of paired stimuli generated within the sequence determine whether the medical treatment device 10 is operated in the first or the second operating mode. Accordingly, to switch the medical treatment device 10 from the first to the second operating mode, the control unit 24 may be configured to increase the rate of isolated second stimuli and thus to decrease the rate of paired stimuli during the course of time. Accordingly, the control unit 24 may be configured to decrease the rate of isolated second stimuli and thus to increase the rate of paired stimuli during the course of time so as to switch the medical treatment device 10 from the second to the first operating mode.

Thereafter, the control unit 24 variedly, i.e. stochastically and/or deterministically and/or combined stochastically-deterministically, determines a duration of the actuating period T_{Ax} in step S3 and a duration of the resting period T_{RX} in step S4. In this context, the term “variedly” means that the determined durations of the actuating periods T_{A1-Ai} and the resting periods T_{R1-Ri} diversely, i.e. non-periodically, vary across the sequence S. The control unit 24 may be configured to, for at least one resting period T_{R1-Ri}, set a duration to 0 s, such that two successively scheduled actuating periods T_{A1-Ai} may directly follow one after another in the sequence S.

For variedly determining the actuating periods T_{A1-Ai} and the resting periods T_{R1-Ri}, the control unit 24 may be configured to stochastically and/or deterministically and/or combined stochastically-deterministically determine the durations. For example, for doing so, the control unit 24 may employ an exponential distribution process and/or a Markov process and/or any other suitable stochastic or deterministic or combined stochastic-deterministic process. In this way, regularities or periodicities in the sequence S may be avoided which may unfavorably interfere with periodicities intrinsic to the pathologically synchronous and oscillatory neuronal activity.

For easier handling, in the following, the sum of an actuating period T_{Ax} and a subsequent resting period T_{Rx} is referred to as an actuation cycle T_{Ax}. It has been found that a small amount of periodicity or repetition in the set of actuation cycles T_{C1-Ci} in the sequence will typically not impair the therapeutic effects of the proposed medical treatment device 10. For example, even if the set of determined actuation cycles T_{C1-Ci} comprises 10% of identical resting period's durations, the proposed medical treatment device 10 may still provide the intended therapeutic effects. However, the control unit 24 may be configured to determine the durations of the resting periods T_{R1-Ri} such that the set of determined actuation cycles T_{C1-Ci} comprises less than 10%, 5% or 1% of identical durations. Alternatively, the control unit 24 may be configured to stipulate or generate the resting periods T_{R1-Ri} such that, in the generated sequence S, the resting periods T_{R1-Ri} or the actuation cycles T_{C1-Ci} have an equal duration.

As set forth above, a number of brain disorders are associated with characteristic abnormal neuronal oscillatory activity in particular frequency bands. For instance, depth recordings in the basal ganglia of patients with Parkinson's disease revealed tremor-related theta band (4 Hz to 7 Hz) activity and bradykinesia-related beta band (9 Hz to 35 Hz) activity. In particular, abnormal neuronal oscillations can be found in different frequency bands.

Accordingly, the control unit 24 may be configured to determine the resting periods T_{R1-Ri} such that a mean frequency of the actuating sequence S does not exceed the upper band edge of the lowest frequency band associated with the brain disease, e.g. 7 Hz in Parkinson patients with tremor. The mean frequency may correspond to or be calculated by the inverse of the sum of a mean actuating periods' duration and a mean resting periods' duration in the sequence S. For example, the resting periods T_{R1-Ri} may be determined such that a mean resting period is in the range between 250 ms and 1000 ms, corresponding to the delta frequency range, i.e. 1 Hz to 4 Hz, or in the range between 140 ms and 250 ms, corresponding to the theta frequency range, i.e. 2 Hz to 7 Hz. Accordingly, the duration of the first and/or second stimuli 14, 18 may be in the range between 30 ms and 250 ms, in particular at around 150 ms.

Alternatively, the control unit 24 may be configured to determine the resting periods T_{R1-Ri} such that the mean frequency of the actuating sequence S is in the range of 5% of the lowest dominant frequency associated with the brain disease or it is up to 2 times or even up to 5 times below the dominant frequency associated with the brain disease.

Then, in steps S5 to S10 or S5 to S10′, the control unit 24 determines which one of the first and second stimuli 14, 18 are to be generated during the actuating period T_{Ax}. These steps, however, differ in dependence on whether paired or isolated stimuli are to be generated.

At first, the generation of paired stimuli is described in the flowing under reference to steps S7 to S10.

In step S7, the control unit 24 is configured to determine for the actuating period T_{A1-Ai} associated to the generation of paired stimuli the number n₁₋ᵢ of first stimuli 14 to be actuated during the respective actuating period T_{A1-Ai}. Specifically, if n equals 1, this means that one first stimulus 14 is generated during the respective actuating period T_{A1-Ai}. In contrast, if n is greater than 1, this means that during the respective actuating period T_{A1-Ai} more than one, i.e. n, first stimuli 14 are generated simultaneously. In the first sequence S₁ depicted in FIG. 2, n₁ and n₅ equal 1; n₂ and n₃ equal 2; n₄ equals 3; and nᵢ equal 4.

More specifically, the control unit 24, across the sequence S of actuating periods T_{A1-Ai} associated to the generation of paired stimuli, is configured to variedly determine the number n₁₋ᵢ of first stimuli 14 to be actuated during the respective actuating period T_{A1-Ai}. In this context, the term “variedly” means that the values for the variable n₁₋ᵢ diversely, i.e. non-periodically, varies across the sequence S.

Specifically, in order to increase variability of stimulus-induced neuronal activations, the control unit 24 may be configured to, for at least one of the actuating periods T_{A1-Ai}, determine one first stimuli 14a-d to be individually actuated during the at least one actuating period T_{A1-Ai}, as depicted in FIG. 2 by actuating period T_{A1} and T_{A5}. In other words, the control unit 24 may be configured to determine for at least one of the variables n₁₋ᵢ a value that equals 1. Additionally or alternatively, the control unit 24 may be configured to, for at least another one of the actuating periods T_{A1-Ai}, determine at least two first stimuli 14a-d to be simultaneously actuated during at least one other actuating period T_{A1-Ai}, as depicted in FIG. 2 by actuating periods T_{A2} and T_{A3}. In other words, the control unit 24 may be configured to determine for at least one of the variables n₁₋ᵢ a value that equals 2. Additionally or alternatively, the control unit 24 may be configured to, for at least another one of the actuating periods T_{A1-Ai}, determine at least three first stimuli 14a-d to be simultaneously generated, as depicted in FIG. 2 by actuating periods T_{A4} and T_{Ai}. In other words, the control unit 24 may be configured to determine for at least one of the variables n₁₋ᵢ a value that is equal to or greater than 3.

For variedly determining the variables n₁₋ᵢ across the sequence S, the control unit 24, for each of the actuating periods T_{A1-Ai} associated to the generation of paired stimuli, is configured to stochastically and/or deterministically and/or combined stochastically-deterministically determine the variables n₁₋ᵢ of first stimuli 14 to be generated i.e. simultaneously generated during the respective actuating periods T_{A1-Ai} of the sequence S. For example, for doing so, the control unit 24 may employ an exponential distribution process and/or a Markov process and/or any other suitable stochastic or deterministic or combined stochastic-deterministic process. In this way, regularities or periodicities in the to sequence S may be avoided, thereby contributing to a robust and effective suppression of pathologically synchronous activity of the patient's neurons during the first operating mode.

In a further development, the process of determining the numbers n₁₋ᵢ of first stimuli 14a-d to be generated may be performed such that values for the number n₁₋ᵢ are provided with an equal probability or with differing probabilities. In this way, the frequency of occurrence for the individual values for the numbers n₁₋ᵢ may be set across the sequence S. For example, in the shown sequence S₁, the values 1 and 2 for the numbers n₁₋ᵢ may be provided with a probability of substantially 33%, respectively, and the values 3 and 4 may be provided with a probability of 16%, respectively. As a result, in the first sequence S₁ having i=6 actuating periods, the values 1 and 2 for n₁₋ᵢ may be determined for two actuating periods, respectively, and the values 3 and 4 for n₁₋ᵢ may be determined for one actuating period, respectively.

Further, the control unit 24 is configured to, across the sequence S of actuating periods T_{A1-Ai}, variedly select the determined number n₁₋ᵢ of different first stimuli 14 from a set of first stimuli comprising the four first stimuli 14a-d, wherein the selected first stimuli 14a-d are to be individually or simultaneously actuated during the respective actuating period T_{A1-Ai}. In this context, the term “variedly” means that the selected first stimuli 14 diversely, i.e. non-periodically, vary across the sequence S. This is performed in step S8 such that, for each actuating period T_{A1-Ai}, each of the plurality of first stimuli 14a-d can only be selected once.

For example, when the control unit 24 has determined for a specific actuating period T_{A1-Ai} that the respective number n₁₋ᵢ equals 1, then the control unit 24 selects a single first stimuli 14 from the set of four first stimuli 14a-d that is to be generated individually during the actuating period T_{Ax}. By contrast, when the control unit 24 has determined that the respective number n₁₋ᵢ equals 2, then the control unit 24 selects two different first stimuli 14 from the set of four first stimuli 14a-d that are to be generated simultaneously during the actuating periods T_{Ax}.

For variedly selecting the first stimuli 14a-d to be generated, the control unit 24, for each of the actuating periods T_{A1-Ai} associated to the generation of paired stimuli, is configured to stochastically and/or deterministically and/or combined stochastically-deterministically select the determined number n₁₋ᵢ of different first stimuli 14 from the set of first stimuli comprising the four first stimuli 14ad. For example, for doing so, the control unit 24 may employ an exponential distribution process and/or a Markov process and/or any other suitable stochastic or deterministic or combined stochastic-deterministic process. In this way, regularities or periodicities in the sequence S may be avoided, thereby contributing to a robust and effective suppression of pathologically synchronous activity of the patient's neurons.

In a further development, each of the first stimuli 14a-d may be provided with an equal probability or with differing probabilities for being selected by the control unit 24. Accordingly, the control unit 24 may select the first stimuli 14a-d in dependence of predefined probabilities for the individual first stimuli 14a-d. In this way, the frequency of occurrence of individual first stimuli 14a-d to being generated may be set across the sequence S. For example, individual first stimuli 14a-d may be provided with a higher relative probability such that they are actuated more frequently during the sequence S.

Additionally or alternatively, the control unit 24 may be configured to select the first stimuli 14a-d for the respective actuating periods T_{A1-Ai} in dependence of a predefined probability or a predefined frequency of occurrence for single first stimuli 14a-d to be individually or exclusively generated during the actuating periods T_{A1-Ai} in the sequence S and/or for a combination of first stimuli 14a-d to be simultaneously generated during the respective actuating periods T_{A1-Ai} in the sequence S. For example, for a combination of two first stimuli 14a-d, a probability or frequency of occurrence may be set as 0 so as to avoid that these two first stimuli 14a-d are generated simultaneously during the sequence S. In other words, the predefined probability or frequency of occurrence may be set so as to prevent single first stimuli 14a-d to be individually or exclusively generated and/or a specific combination of first stimuli 14a-d to be simultaneously generated in sequence S. In a further example, a probability or frequency of occurrence for specific single first stimuli 14a-d to be individually generated and/or for specific combinations of first stimuli 14a-d to be simultaneously generated may be set relatively high such that they are generated more frequently during the sequence S. Further, the predefined probabilities or frequencies of occurrence may vary during the course of the sequence S.

When paired stimuli are to be generated, the control unit 24 is configured to actuate the second stimulating device 16 so as to generate the second stimuli 18a-d to be paired to the generation of at least a part of the first stimuli 14. In the medical treatment device 10, to each one of the first stimuli 14a-d at least one specific second stimuli 18a-d is associated such that it is predefined which second stimuli 18 are to be paired or can be paired to the respective first stimuli 14. In the embodiment shown in FIGS. 2 and 3, to each first stimuli 14a-d a single second stimuli 18a-d is associated. Specifically, to the first stimuli 14a the second stimuli 18a, to the first stimuli 14b the second stimuli 18b, to the first stimuli 14c the second stimuli 18c, and to the first stimuli 14d the second stimuli 18d are associated. Accordingly, by determining the first stimuli to be generated during the respective actuating period T_{A1-Ai}, the control unit 24, due to the predefined correlation between the first and second stimuli 14, 18, also determines which one of the second stimuli 18 are to be generated during the respective actuating period T_{A1-Ai}.

In a further development, to each one of the first stimuli, a different set of second stimuli may be associated such that each second stimuli to be paired to a respective first stimulus is selected from the set of second stimuli associated to the respective first stimulus. In this way, redundancies during the medical treatment may be avoided, thereby preventing that the patient perceives the treatment as annoying and/or boring.

The sets of second stimuli may be provided such that the second stimuli 18 differ among the sets of second stimuli. This may enable an efficient pairing. For example, when the second stimulating device 16 is provided in the form of an acoustic stimulator, the second stimuli 18 may be provided in the form of different sets of tones, which may be disjoint such that each tone of the sets of tones is assigned to only one first stimulus 14. Specifically, the control unit 24, for paring second stimuli 18 to a first stimuli 14, may be configured to stochastically and/or deterministically and/or combined stochastically-deterministically select at least one second stimulus from the set of second stimuli associated to the respective first stimulus. For example, for doing so, the control unit 24 may employ an exponential distribution process and/or a Markov process and/or any other suitable stochastic or deterministic or combined stochastic-deterministic process.

In the flowing, the generation of isolated stimuli is described in the flowing under reference to steps S7′ to S10′.

In step S7′, the control unit 24 is configured to determine for the actuating period T_{A1-Ai} associated to the generation of isolated stimuli the number u₁₋ᵢ of second stimuli 18 to be actuated during the respective actuating period T_{A1-Ai}. Specifically, if u equals 1, this means that one second stimulus 18 is generated during the respective actuating period T_{A1-Ai}. In contrast, if u is greater than 1, this means that during the respective actuating period T_{A1-Ai} more than one, i.e. u, second stimuli 18 are generated simultaneously. In the second sequence S₂ depicted in FIG. 2, u₁ equals 2; u₃ and u₄ equal 3; and uᵢ equal 4.

More specifically, the control unit 24, across the sequence S of actuating periods T_{A1-Ai} associated to the generation of isolated stimuli, is configured to variedly determine the number u₁₋ᵢ of second stimuli 18 to be actuated during the respective actuating period T_{A1-Ai}. In this context, the term “variedly” means that the values for the variable u₁₋ᵢ diversely, i.e. non-periodically, varies across the sequence S.

Specifically, in order to increase variability of stimulus-induced neuronal activations, the control unit 24 may be configured to, for at least one of the actuating periods T_{A1-Ai}, determine one second stimuli 18a-d to be individually actuated during the at least one actuating period T_{A1-Ai}. In other words, the control unit 24 may be configured to determine for at least one of the variables u₁₋ᵢ a value that equals 1. Additionally or alternatively, the control unit 24 may be configured to, for at least another one of the actuating periods T_{A1-Ai}, determine at least two second stimuli 18a-d to be simultaneously actuated during at least one other actuating period T_{A1-Ai}, as depicted in FIG. 3 by actuating period T_{A1}. In other words, the control unit 24 may be configured to determine for at least one of the variables u₁₋ᵢ a value that equals 2. Additionally or alternatively, the control unit 24 may be configured to, for at least another one of the actuating periods T_{A1-Ai}, determine at least three second stimuli 18a-d to be simultaneously generated, as depicted in FIG. 3 by actuating periods T_{A3}, T_{A4} and T_{Ai}. In other words, the control unit 24 may be configured to determine for at least one of the variables u₁₋ᵢ a value that is equal to or greater than 3.

For variedly determining the variables u₁₋ᵢ across the sequence S, the control unit 24, for each of the actuating periods T_{A1-Ai} associated to the generation of isolated stimuli, is configured to stochastically and/or deterministically and/or combined stochastically-deterministically determine the variables u₁₋ᵢ of second stimuli 18 to be generated i.e. simultaneously generated during the respective actuating periods T_{A1-Ai} of the sequence S. For example, for doing so, the control unit 24 may employ an exponential distribution process and/or a Markov process and/or any other suitable stochastic or deterministic or combined stochastic-deterministic process. In this way, regularities or periodicities in the sequence S may be avoided, thereby contributing to a robust and effective suppression of pathologically synchronous activity of the patient's neurons.

In a further development, the process of determining the numbers u₁₋ᵢ of second stimuli 18a-d to be generated may be performed such that values for the number u₁₋ᵢ are provided with an equal probability or with differing probabilities. In this way, the frequency of occurrence for the individual values for the numbers u₁₋ᵢ may be set across the sequence S.

Further, the control unit 24 is configured to, across the sequence S of actuating periods T_{A1-Ai}, variedly select the determined number u₁₋ᵢ of different second stimuli 18 from a set of second stimuli comprising the four second stimuli 18a-d, wherein the selected second stimuli 18a-d are to be individually or simultaneously actuated during the respective actuating period T_{A1-Ai}.

For variedly selecting the second stimuli 18 a-d to be generated, the control unit 24, for each of the actuating periods T_{A1-Ai} associated to the generation of isolated stimuli, is configured to stochastically and/or deterministically and/or combined stochastically-deterministically select the determined number u₁₋ᵢ of different second stimuli 18 from the set of second stimuli comprising the four second stimuli 18a-d. For example, for doing so, the control unit 24 may employ an exponential distribution process and/or a Markov process and/or any other suitable stochastic or deterministic or combined stochastic-deterministic process. In this way, regularities or periodicities in the sequence S may be avoided, thereby contributing to a robust and effective suppression of pathologically synchronous activity of the patient's neurons.

In a further development, each of the second stimuli 18a-d may be provided with an equal probability or with differing probabilities for being selected by the control unit 24. Accordingly, the control unit 24 may select the second stimuli 18a-d in dependence of predefined probabilities for the individual second stimuli 18a-d. In this way, the frequency of occurrence of individual second stimuli 18a-d to being generated may be set across the sequence S.

Additionally or alternatively, the control unit 24 may be configured to select second stimuli 18a-d for the respective actuating periods T_{A1-Ai} in dependence of a predefined probability or a predefined frequency of occurrence for single second stimuli 18a-d to be individually or exclusively generated during the actuating periods T_{A1-Ai} in the sequence S and/or for a combination of second stimuli 18ad to be simultaneously generated during the respective actuating periods T_{A1-Ai} in the sequence S.

As set forth above, the above described steps S2 to S10 or S2 to S10′ are performed repeatedly for each one of the actuating periods T_{A1-Ai} within the sequence S to be generated.

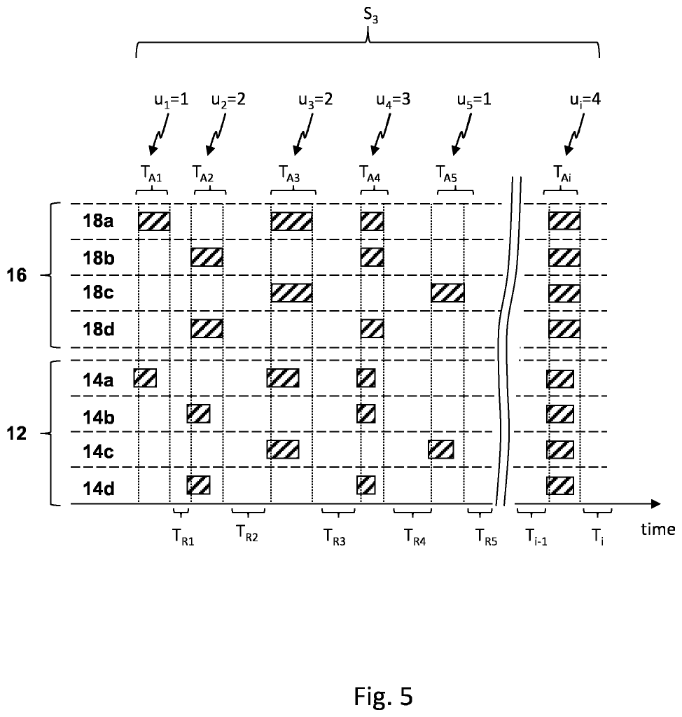

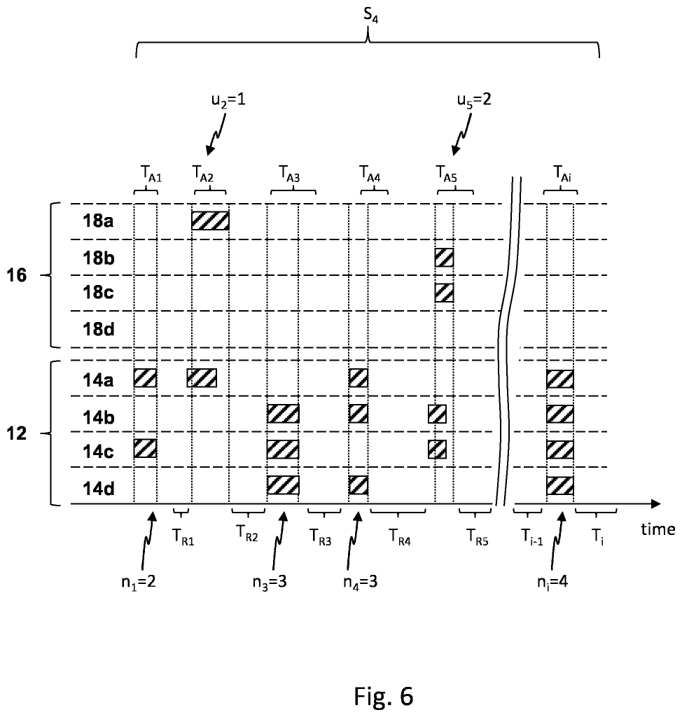

In a further development, the control unit 24 may be configured to further operate the medical treatment device 10 in a third and fourth operating mode. Specifically, the third operating mode differs from the first operating mode in that the role of the first stimulating device 12 and the second stimulating device 16, i.e. and thus the role of the first stimuli 14 and the second stimuli 18, are switched, as can be gathered from FIGS. 5 and 6 depicting corresponding sequences S₃, S₄ of actuating periods of the the third and fourth operating mode, respectively. Specifically, in this configuration, the control unit 24 is configured to operate the first and the second stimulating device 12, 16 successively in the first operating mode, the second operating mode, the third operating mode and the fourth operating mode. This may also be referred to as a criss-cross pairing mode of the medical treatment device 10.

In this configuration, the third operating mode corresponds to a “learning phase”. Thus, administrating paired first and second stimuli 14, 18 to the patient's body may have the effect that the nerve system of the patient is conditioned and/or associatively learns to react to the first stimuli 14 in the same way or in a slightly attenuated form as to the second stimuli 18 even when the second stimuli 18 are no longer administered to the patient's body, e.g. during the fourth operating mode. In this way, the conditioning and thus the efficiency of the treatment procedure may further be improved.

Accordingly, in this configuration, the second stimuli 18 are therapeutically effective sensoric stimuli and thus are also referred to as “specific” stimuli. In other words, the second stimuli 18 are configured to, when being administrated to the patient's body without applying conditioning and/or associative learning approaches or procedures, suppress the pathologically synchronous activity of neurons.

Specifically, in the third operating mode, the control unit 24 may be configured to actuate the second stimulating device 16 in the third sequence S₃ of successive actuating periods T_{A1-Ai} such that a number u of first stimuli 14 to be generated during the actuating periods T_{A1-Ai} is variedly determined across the third sequence S₃ and to actuate the first stimulating device 12 so as to generate the first stimuli 14 to be paired to the generation of at least a part of the second stimuli 18. In the fourth operating mode, the control unit 24 may be configured to actuate the first stimulating device 12 so as to generate the first stimuli 14 to be de-coupled from the generation of at least a part of the second stimuli 18. The sequences S_{3,4} of actuating periods T_{A1-Ai} of the third and fourth operating mode may be generated based on the procedure depicted in FIG. 4, wherein the role of the first and second stimuli 14, 18, together with the variables n and u, are switched throughout the above described procedure. It will be obvious for a person skilled in the art how to perform the procedure for generating the third and fourth sequence S₃, S₄. Thus, a corresponding description thereof is omitted in order to avoid redundancies.

In a further development, the medical treatment device 10 may further comprise a sensor unit 28 for measuring stimulating effects on the affected neurons. This sensor unit 28 is depicted in FIG. 1 by means of dashed lines and constitutes an optional component of the medical treatment device 10. In this configuration, the sensor unit 28 is connected to the control unit 24, i.e. wirelessly or by means of connecting wires, so as to transfer measured data to the control unit 24. For example, the sensor unit 28 may be comprised in a mobile device and may be configured to obtain disease-related scores and/or life quality scores.

Specifically, the control unit 24 may be configured to operate the first and the second stimulating device 12, 16 in the respective operating modes in dependence on data measured by the sensor to unit 28. Further, the control unit 24 may be configured to switch the first and the second stimulating devices 12, 16 from the second operating mode into the first operating mode when a value indicative of the stimulating effects on the neurons measured by the sensor unit 28 reaches a predefined threshold value. Alternatively or additionally, the medical treatment device 10 may be configured such that a patient to be treated may manually switch between the operating modes in a demand-controlled manner.

Further, the control unit 24 may be configured to adapt the first and second stimuli 14, 18 generated by the first and second stimulating devices 12, 16 and/or the respective sequences S of actuating periods T_{A1-Ai} in dependence on data measured by the sensor unit 24.

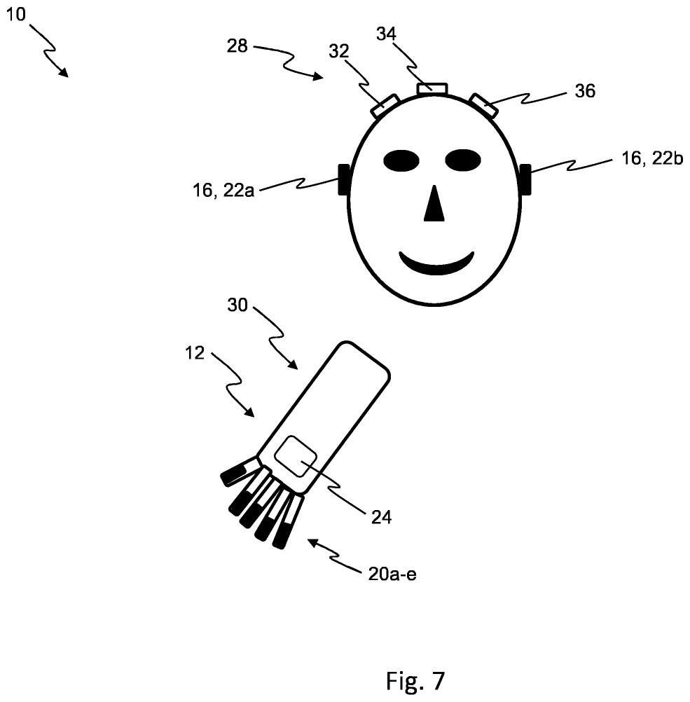

FIG. 7 shows another embodiment of the medical treatment device 10, in which the first stimulating device 12 is provided in the form of a medical treatment glove 30 fastened to a right hand of a patient and the second stimulating device 16 is an acoustic stimulator, i.e. provided in the form of earphones fastened to the patient's ears. The medical treatment device 10 further comprises a sensor unit 28 which is wirelessly connected to the control unit 24.

The medical treatment glove 30 comprises five first stimulating units 20a-e fastened to different fingers, i.e. fingertips, of the patient's hand. The stimulating units 20a-e may comprise qualitatively different mechanical stimulators for actuating receptors in the patient's hand. For example, the first stimulating units 20a-e may comprise piezo vibrators, a linear motor and/or a voice coil.

In general, the human skin comprises mechanoreceptive afferent units capable of sensing stimuli, i.e. tactile or vibratory stimuli, which have been classified into two major categories, namely into fast adapting units (FA) and slowly adapting units (SA). The FA units respond to moving stimuli as well as the onset and removal of a step stimulus. In contrast, the SA units respond with a sustained discharge. In addition, based on the properties of their receptive fields, both categories are further classified into two different types. The fast-adapting type I (FA I) units, also referred to as RA (rapidly adapting) units, and the slow-adapting type I (SA I) units form a small, but clearly delimited receptive fields on the surface of the skin. In contrast, the receptive fields formed by the fast-adapting type II (FA II) units, also referred to as PC (Pacinian corpuscles) units, and the slow-adapting type II (SA II) are wider and have obscure borders.

Typically, the distribution and density of the different types of mechanoreceptors differs in dependence on the position on the human skin. For example, regarding the glabrous skin of the human hand, the density of FA I units is relatively high in an area of the fingertips. By contrast, the density of FA II units is relatively high in an area at the back of the fingers and the hand.

The four different types of human cutaneous mechanoreceptors respond optimally to qualitatively different stimuli. Specifically, edge stimuli and stretch stimuli are optimal for SA I and SA II mechanoreceptors, respectively. SA I units often have a rather irregular sustained discharge, whereas SA II units discharge in a regular manner, but often display spontaneous discharge in the absence of tactile stimulation. Vibratory perpendicular sinusoidal skin displacements in the range between about 30 Hz to about 60 Hz are optimal stimuli for FA I units, whereas vibratory stimuli in the range between about 100 Hz to about 300 Hz are optimal stimuli for FA II units. FA I and, especially, SA I units have a pronounced edge contour sensitivity and, hence, their response is stronger when a stimulating contactor surface which is not completely contained in the receptive field. Accordingly, to enhance the FA I responses, instead of a flat, spatially homogenous contactor surface of the stimulation element one could use a contactor surface with a spatially inhomogeneous indentation profile.

In the shown embodiment, the first stimulating units 20a-e may be designed and configured to generate stimuli adapted to the response characteristic of FA I, FA II, SA I and/or SA II units. In this configuration, each of the first stimulating units 20a-e may be configured to generate first stimuli 14 adapted to response to at least one of the FA I, FA II, SA I and SA II units. For example, the stimulating units 20a-e may be configured to generate first stimuli 14 which target merely one of the FA I, FA II, SA I and SA II units. In other words, these first stimulating units 20a-e generate first stimuli 14 which are adapted to the response characteristic of one of the FA I, FA II, SA I and SA II units. Alternatively or additionally, the first stimulating units 20a-e may target more than one FA I, FA II, SA I and SA II units. For example, such a first stimulating unit 20a-e may be configured to generate first stimuli 14 which are sensed by more than one of the FA I, FA II, SA I and SA II units. Alternatively or additionally, such a stimulating unit 20a-e may be configured to being operated in different operational modes, in which different first stimuli 14 are generated which, respectively, are adapted to a response to characteristic of different FA I, FA II, SA I and SA II units.

Specifically, for targeting FA I type receptors, a first stimulating unit 20a-e may be configured to generate vibratory stimuli with a vibration frequency between 30 Hz to 60 Hz, i.e. 30 Hz, and a vibration peak-to-peak amplitude of 0.25 mm. For example, this first stimulating unit 20a-e may be intended to being fastened to a fingertip of the patient. Further, for targeting FA II type receptors, a first stimulating unit 20a-e may be configured to generate vibratory stimuli with a vibratory frequency between 100 Hz to 300 Hz, i.e. 250 Hz, and a peak-to-peak amplitude between 0.015 mm and 0.8 mm, e.g. between 0.015 mm to 0.0.2 mm. For example, this first stimulating unit 20a-e may be into tended to being fastened to a back of a finger or hand of the patient. Further, it has been found that for sufficiently large vibration peak-to-peak amplitudes, the low-frequency vibration targeting FA I type receptors will additionally activate FA II type receptors and vice versa. Thus, by increasing the peak-to-peak amplitude, e.g. to a peak-to-peak amplitude of 3.0 mm, each of the above mentioned first stimulating units 20a-e may generate vibratory stimuli adapted to stimulate both FA I and FA II type receptors.

The second stimulating device 16 may be configured to generate non-specific, acoustic second stimuli 18 in the form of music, tunes, natural sounds, i.e. in a natural or processed way, and/or sequences of tones of non-dissonant scales, e.g. pentatonic scales. Specifically, the second stimulating device 16 may provide unilateral or bilateral acoustic second stimuli 18. Typically, sound duration of the second stimuli 18 ranges between 20 ms and 250 ms, in particular 150 ms, wherein the intensities of the therapeutic tones are set to be comfortable for the patient, i.e. having a level of 5 dB or any other comfortable dB level, which optionally can be adjusted by the patient, i.e. to the ambient noise level.

As set forth above, the control unit 24 is provided for selectively and intermittently actuating the first and the second stimulating device 12, 16, i.e. the first stimulating units 20 and the second stimulating units 22. In this way, the control unit 24 controls the generation of the first and second stimuli. More specifically, the control unit 24 may be configured to adapt or set the characteristic of the first and the second stimuli, e.g. in terms of stimuli duration, stimuli strength, stimuli frequency and/or stimuli time course.

For example, the stimuli to be generated by the first and/or the second stimulating device 12, 16 may be specified based on an amplitude curve which refers to a time course of stimuli strength. In this context, the stimuli may be generated with different waveforms, e.g. when being provided in the form of mechanical stimuli or vibrations. In particular, the control unit 24 may be configured to variedly set a waveform of the different stimuli among the sequence of actuating periods and/or among the respective actuating periods. For example, for doing so, the control unit may employ an exponential distribution process and/or a Markov process and/or any other suitable stochastic or deterministic or combined stochastic-deterministic process. Specifically, the stimuli may be generated so as to be provided in the form of sine waves or trapezoidal waves.

It has been recognized that, since the different waveforms may have different power spectra, different waveforms may activate proprioceptive receptors differently. As to substance, the spectrum of a trapezoidal waveform may contain higher frequency components. Accordingly, given the known tuning characteristics of the above described RA (rapidly adapting) units and the above described PC (Pacinian corpuscles) units, a 30 Hz vibration with a sine wave at sufficiently small vibration amplitude may activate, i.e. predominantly activate, receptors of the RA units, also referred to as the fast-adapting type I (FA I) units. Further, a 30 Hz vibration with a trapezoidal wave-form having substantially corresponding or identical vibration amplitudes compared to the sine wave may additionally activate receptors of the PA units, also referred to as the fast-adapting type II (FA II) units.

Accordingly, to vary the extent and composition of the neuronal subpopulations stimulated by the different vibratory stimuli delivered to the same part of the body, e.g. to the same fingertip, the stimulus waveform, in particular within a sequence, may be varied, e.g. from one stimulus to another and in particular in a deterministic or stochastic or combined deterministic-stochastic manner.

The sensor unit 28 may be optional and may comprise at least one non-invasive sensor. For example, it may comprise sensors for acquiring Electroencephalography (EEG) recordings (assessing brain activity), Magnetoencephalography (MEG) recordings (assessing brain activity), Electromyography (EMG) recordings (assessing muscular activity, e.g. tremor). Further, the sensor unit 28 may comprise sensors for registering kinematic parameters, such as accelerometers (to measure tremor or amount of movement production).

Alternatively or additionally, the sensor unit 28 may comprise at least one invasive sensor. For example, such an invasive sensor may be provided in the form of electrodes, e.g. epicortical, epidural, intracortical or depth electrodes, configured to be implanted in the patient's brain, in order to provide signals, in particular local field potentials (LFP), generated by active neurons. A less invasive alternative are subcutaneous electrodes, i.e. electrode implanted under the skin of the head.

In the shown embodiment, for measuring stimulation effects and neuronal activities, the sensor unit 28 comprises two non-invasive EEG electrodes 32, 34 connected to a controller 36 of the sensor unit 28. In an alternative embodiment, the sensor unit 28 may alternatively or additionally comprise invasive electrodes (not shown), e.g. epicortical electrodes. The controller 36 amplifies and analyzes the signals provided by the electrodes 32, 34 and wirelessly transmits the thus acquired information to the control unit 24. In an alternative embodiment, the control unit 24 may be connected to the controller 36 of the sensor unit 28 via a connecting wire.

More specifically, the control unit 24 may be configured to adapt characteristics of the first and second stimuli 14, 18, e.g. in terms of stimuli duration, stimuli strength, stimuli frequency and/or stimuli time course, in dependence of the information or data acquired by the sensor unit 28. For example, in case the sensor unit 28 detects or measures increased levels of disease-related spectral power in EEG, MEG, EMG or LFP recordings, the control unit 24 may be configured to respectively increase stimulation intensity by increasing the rate of paired first and second stimuli 14, 18.

Further, the control unit 24 may be configured to iteratively adapt the characteristics of the first and second stimuli 14, 18 in dependence of the information or data acquired by the sensor unit 28. In particular, the control unit 24 may be configured to analyze the acquired data of the sensor unit 28 so as to selectively adapt the characteristics of the first and second stimuli 14, 18. For example, the control unit 24 may perform a spectral analysis based on EEG, MEG, EMG and/or LFP recordings acquired by the sensor unit 28. Then, over the duration of one or more treatment procedures performed by the medical treatment device 10, the control unit 24 may be configured to register changes of brain activity, in particular, changes of spectral power in disease-related frequency bands (e.g. theta and/or beta band in Parkinson's disease) caused by stimulating the patient's body by means of the first and second stimulating device 12, 16. Thereafter, characteristics of the first and second stimuli 14, 18 are stepwise or iteratively changed so as to cause changes of the spectral power and to track the changes by means of the sensor unit 28. For example, the control unit 24 may be configured to increase or decrease the rate of paired first and second stimuli 14, 18. In this way, the control unit 24 may automatically identify and adapt relevant characteristics or parameters of the first and second stimuli 14, 18 which cause most pronounced reduction of disease related spectral power.

Further, the information or data acquired by the sensor unit 28 may be used by the control unit 24 to adapt the mean frequency of the actuating sequence, i.e. by respectively changing the resting periods T_{R1-Ri} in the sequences S. For example, the control unit 24 may perform a spectral analysis based on EEG, MEG, EMG and/or LFP recordings acquired by the sensor unit 28 so as to determine dominant oscillatory frequency components. Based thereupon, the control unit 24 may be configured to adapt the mean frequency of the actuating sequences S such that it is in the range of ±5% of the lower bound of the lowest dominant frequency of the feedback signal, or it is at the lower edge of the lowest dominant frequency of the feedback signal, or it is up to 2 times or even up to 5 times below the dominant frequency of the feedback signal.

Additionally or alternatively, the control unit 24 may be configured to, in response to the acquired data or information by the sensor unit 28, generate a warning signal for the patient, the warning signal being indicative of, for example, increasing a daily treatment duration. Accordingly, the medical treatment device 10 may comprise a means for outputting the warning signal, e.g. a display or a transmitting unit. Specifically, the transmitting unit may be configured to output the warning signal to a mobile device, such as a mobile phone, of the patient capable of displaying the warning signal to the patient.

In the shown medical treatment device 10, the first and the second stimulating device 12, 16 are operated repeatedly in the above described first and second operating modes.

In a further development, the medical treatment device 10 may be configured to output a further signal indicative of a change of operating mode of the medical treatment device 10 to the mobile device, i.e. a smartphone, and/or an external device for generating a warning signal, i.e. an acoustic warning signal or an visual warning signal, for informing and/or instructing the patient. For example, when the control unit 24 is configured to actuate merely the second stimulating device 16 during the second operating mode, i.e. without actuating the first stimulating device 12, the further signal may indicate to the patient that the first stimulating device 12 in the form of the medical treatment glove 30 should be attached to its hand when the medical treatment device 10 is to be operated in the first operating mode and to detach the medical treatment glove 30 from its hand when the medical treatment device 10 is to be operated in the second operating mode. In this way, the patient is instructed to wear the medical treatment glove 30 only when the medical treatment device 10 is operated in the first operating mode. As a result, a more convenient medical treatment for the patient may be provided.

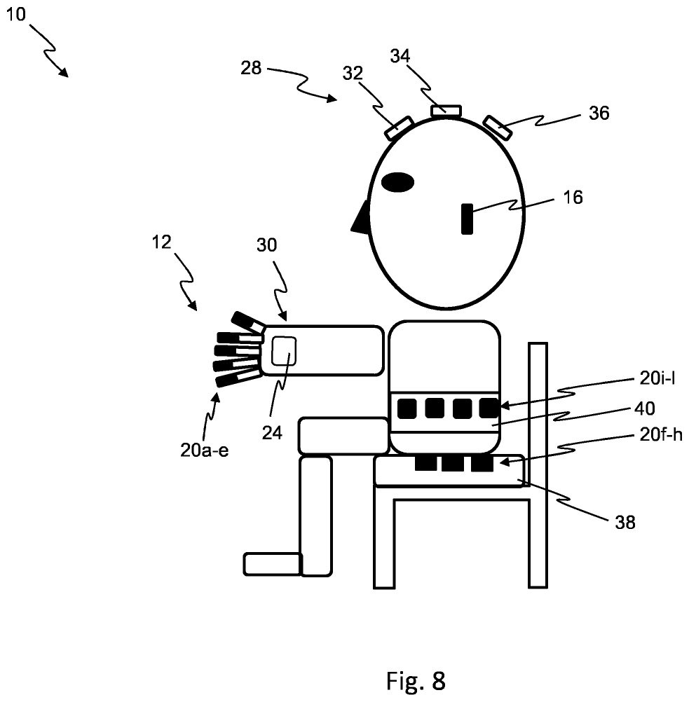

FIG. 8 schematically shows a further embodiment of the medical treatment device 10 which differs from the embodiment depicted in FIG. 7 in that the first stimulating device 12 further comprises a vibratory seat pad 38 and a vibratory belly band 40, each of which accommodates further first stimulating units 20 for generating first stimuli 14. Thus, technical features that are previously described may also relate and be applied to this embodiment of the medical treatment device 10. In an alternative embodiment, the first stimulating device 12 may additionally or alternatively comprise a vibratory neck band and vibratory shoulder band.

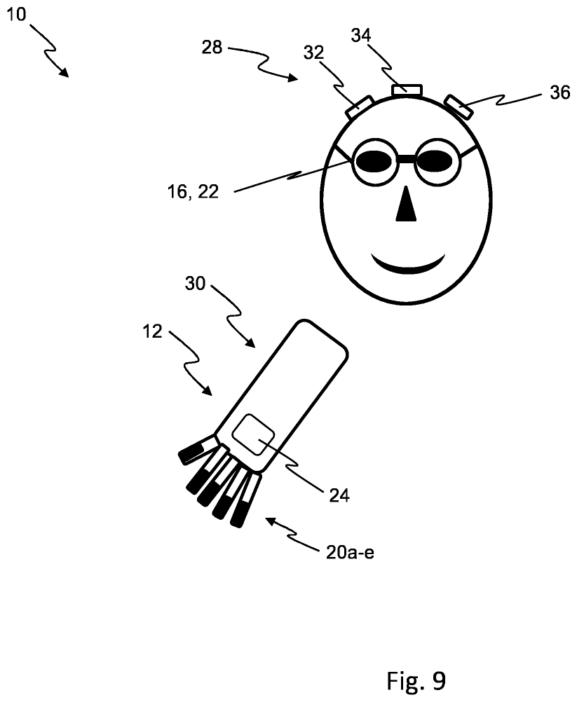

FIG. 9 schematically shows a further embodiment of the medical treatment device 10 which differs from the embodiment depicted in FIG. 7 in that the second stimulating device 16 is provided to in the form of a visual stimulator. The visual stimulator may generate optical or visual second stimuli 18 by providing visual stimulation pattern. Specifically, the visual stimulator is provided in the form of medical treatment glasses positioned in front of the patient's eyes. The visual stimulator is capable of manipulating light sensed by the patient's eyes. This may be realized by selectively controlling brightness and/or intensity and/or transparency of different segments of visual fields of the glasses so as to generate the second stimuli 18. For example, for doing so, the glasses may comprise transmission glasses which may change their transmission properties, i.e. transparency, in different segments separately, i.e. by setting a current or voltage, ranging from a maximal transmission, i.e. between 60% and 100%, to a minimal transmission, i.e. in the range of 0% to 30%. Alternatively and additionally, the glasses may comprise arrays of LEDs or other means for light emission with maximum light intensities, for example, in the range of 1 cd/cm² to 20 cd/cm². Alternatively or additionally, the glasses may comprise video and/or movie displays with segment light intensity variation with maximum intensities, for example, in the range of 60% to 100% and minimum intensities, for example, in the range of 0% to 30%.

In this configuration, the medical treatment device 10 is configured to, when being operated in the second operating mode, to actuate both the first and the second stimulating device 12, 16 such that unpaired first and unpaired second stimuli 14, 18 are generated. In this state, the first and the second stimuli 14, 18 do not overlap.

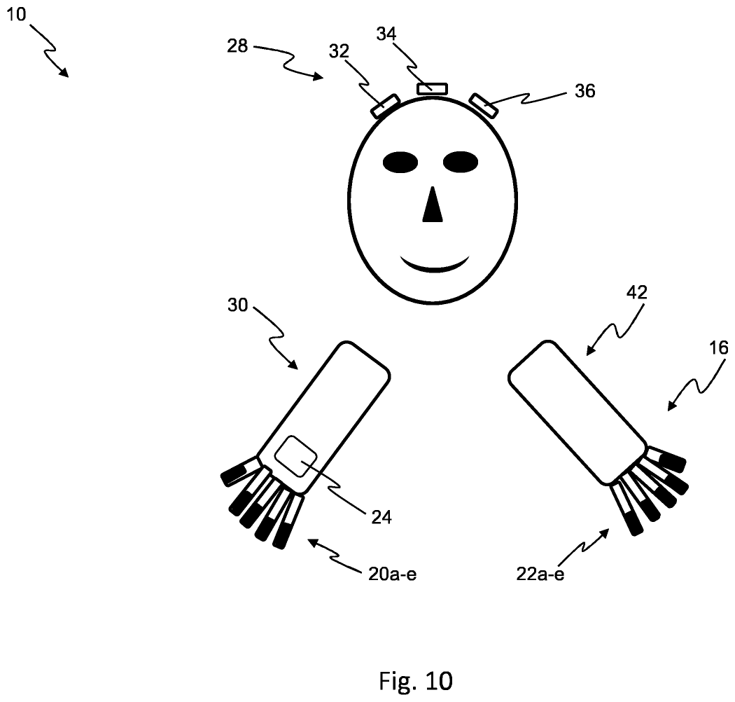

FIG. 10 schematically shows a further embodiment of the medical treatment device 10 which differs from the embodiment depicted in FIG. 7 in that both the first and the second stimulating device 12, 16 are configured to generate specific stimuli. Thus, technical features that are previously described may also relate and be applied to this embodiment of the medical treatment device 10. Specifically, the second stimulating device 16 is provided in the form of a further medical treatment glove 42 fastened to the other hand of the patient compared to the treatment glove 30. The second stimulating device 16 comprises five second stimulating units 22a-e wirelessly connected to the control unit 24 for generating different specific second stimuli 18, wherein configuration and design of the second stimulating units 22a-e correspond to those of the first stimulating units 20a-e.

In this configuration, the control unit 24 is configured to operate the first and the second stimulating device 12, 16 successively in the above described first operating mode, second operating mode, third operating mode and fourth operating mode. In this way, the medical treatment device 10 is employed in the so-called criss-cross pairing mode.

More specifically, during the first operating mode, the second stimuli 18 generated by the second stimulating device 16 are paired to the first stimuli 14 of the first stimulating device 12. In this way, after successfully pairing both stimuli during the first operating mode, vibratory fingertip stimulation of the left hand during the second operating mode by means of the further medical treatment glove 42 will, on average, have an effect that is similar, but not identical, to the bilateral vibratory fingertip stimulation using both stimulating devices 12, 16, i.e. medical treatment gloves 30, 42.

Then, to further increase the efficiency of the medical treatment, the control unit 24 switches the first and the second stimulating device 12, 16 into the third operating mode and thereafter into the fourth operating mode, in which the roles between the first and the second stimulating device 12, 16 and between the first and the second stimuli 14, 18 are switched, such that the former first stimuli becomes the second stimuli and vice versa. Accordingly, in the third operating mode, a pairing of the first stimuli 14 generated by the first stimulating device 12 to the second stimuli 18 of the second stimulating device 16 is performed. As a result, when being operated thereafter in the fourth or second operating mode, the bilateral stimulation effect of unilateral stimulation, e.g. vibratory fingertip stimulation of the right hand, is even closer to the effects of bilateral stimulation, i.e. vibratory fingertip stimulation of the right and the left hand, as opposed to without the criss-cross pairing. In this way, the mutual conditioning of both stimuli may be improved by the criss-cross pairing mode.

Specifically, the criss-cross operating mode may be performed when the control unit 24 determines that the operating time of the medical treatment device 10 in the first operating mode is greater compared to the operating time of the second operating mode, i.e. which may be an indicator of inefficient pairing or conditioning. In this case, the criss-cross pairing may be performed to test whether a reverse pairing may have a greater potential for conditioning. Further, the control unit 24 may be configured to operate the medical treatment device 10 in the criss-cross operating mode so as to boost the mutual conditioning of the first and second stimuli. For doing so, for example, the controller 24 may be configured to employ the medical treatment device 10 in the criss-cross operating mode, when the operating time in the first operating mode reaches a threshold value, which may be in particular a value between 10% and 50%, i.e. 10%, 20%, 30%, 40%, or 50%, of a total operating time of the medical treatment device 10. Alternatively or additionally, the controller 24 may be configured to operate the medical treatment device 10 in the third and fourth operating mode when the control unit 24 determines that the medical treatment device 10 has been switched from the second into the first operating mode for a predefined number N of times, wherein N is an integer which may range from the 3 to 50.

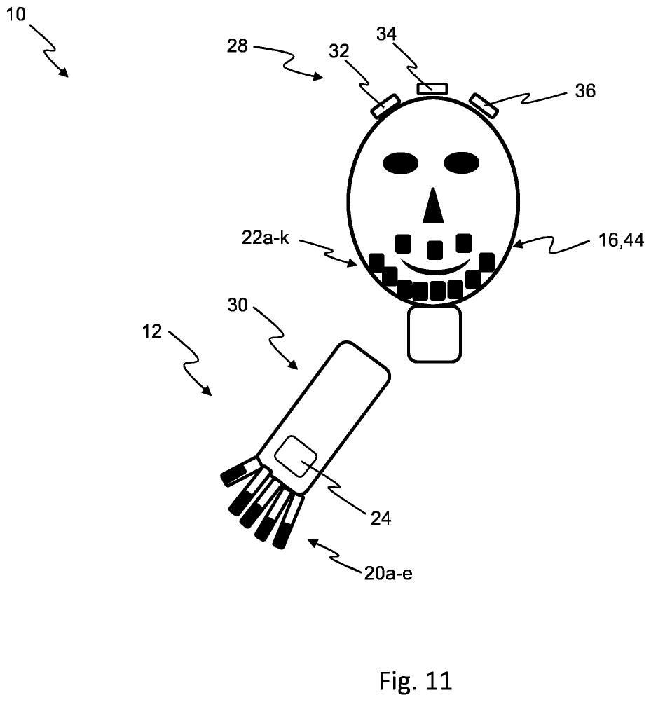

In a further embodiment of the medical treatment device 10 depicted in FIG. 10, the first and/or the second stimulating unit 20, 22 may be provided in the form of or may further comprise a vibratory face mask, a vibratory voice box, vibratory belly band, and/or vibratory seat pad. In this context, FIG. 11 schematically shows a further embodiment of the medical treatment device 10 which differs from the embodiment depicted in FIG. 10 in that the second stimulating device 16 is a vibratory face mask 44 having a plurality of second stimulating units 22 for inducing vibratory second stimuli 18 to the patient's face. Thus, technical features that are previously described may also relate and be applied to this embodiment of the medical treatment device 10.

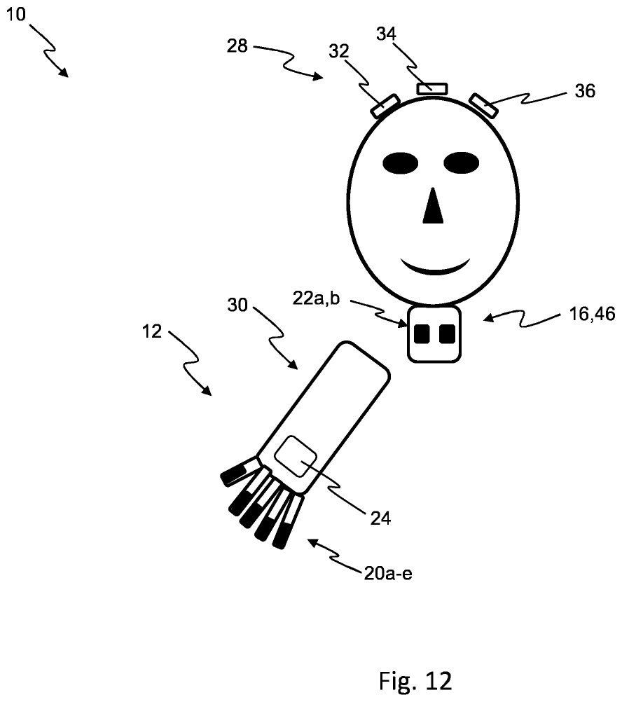

FIG. 12 schematically shows a further embodiment of the medical treatment device 10 which differs from the embodiment depicted in FIG. 10 in that the second stimulating device 16 is a vibratory voice box 46 having a plurality of second stimulating units 22 for inducing vibratory second stimuli 18 to the patient's voice box. Thus, technical features that are previously described may also relate and be applied to this embodiment of the medical treatment device 10.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention.

### LIST OF REFERENCE NUMERALS

10 medical treatment device
12 first stimulating device
14 first stimuli
16 second stimulating device
18 second stimuli
20 first stimulating units
22 second stimulating units
24 control unit
26 connecting line
28 sensor unit
30 medical treatment glove
32, 34 electrodes
36 controller
38 medical treatment seat pad
40 medical treatment belly band
42 further medical treatment glove
44 medical treatment face mask
46 medical treatment voice box

1. Medical treatment device for stimulating neurons of a patient to suppress a pathologically synchronous activity of the neurons, the device comprises:
a first non-invasive stimulating device for generating at least two different first stimuli to a patient's body,
a second non-invasive stimulating device for generating at least two different second stimuli to the patient's body, and
a control unit for selectively and intermittently actuating the first and the second stimulating device, wherein the control unit is configured to,
in a first operating mode, actuate the first stimulating device in a sequence of successive actuating periods such that a number n of first stimuli to be generated simultaneously during the actuating periods is variedly determined across the sequence and to actuate the second stimulating device so as to generate the second stimuli to be paired to the generation of at least a part of the first stimuli, and
in a second operating mode, to actuate the second stimulating device so as to generate the second stimuli to be de-coupled from the generation of at least a part of the first stimuli.

2. Medical treatment device according to claim 1, wherein the first and the second stimuli differ from one another, in particular in terms of stimulation modality and/or stimuli strength and/or stimuli frequency and/or stimuli time course and/or location of origin and/or stimulating unit of origin.

3. Medical treatment device according to claim 1 or 2, wherein the first and the second stimuli may comprise at least one modality of mechanical stimulus, in particular tactile stimulus and/or vibratory stimulus, and/or electrical stimulus and/or optical stimuli and/or acoustic stimuli and/or chemical stimuli and/or thermal stimuli, and in particular the first stimuli comprises at least one of mechanical stimulus and/or electrical stimulus.

4. Medical treatment device according any one of claims 1 to 3, wherein the first and/or the second stimulating device are/is configured to generate first or second stimuli to different sites of the patient's body.

5. Medical treatment device according any one of claims 1 to 4, wherein the first stimulating device comprises a plurality of first stimulating units, each of which generates at least one of the first stimuli, and/or
the second stimulating device comprises a plurality of second stimulating units, each of which generates at least one of the second stimuli.

6. Medical treatment device according to any one of claims 1 to 5, wherein the first stimuli are configured to, when being administrated to the patient's body, suppress the pathologically synchronous activity of neurons, in particular in at least one of a patient's brain or spinal cord.

7. Medical treatment device according to any one of claims 1 to 6, wherein the second stimuli are configured to, when being administrated to the patient's body, suppress the pathologically synchronous activity of neurons, in particular in at least one of a patient's brain or spinal cord.

8. Medical treatment device according to any one of claims 1 to 7, wherein the control unit is configured to operate the first and the second stimulating device in the first operating mode and subsequent in the second operating mode.

9. Medical treatment device according to any one of claims 1 to 8, wherein in the second operating mode, the first and the second operating device are actuated such that the generation of the second stimuli takes place isolated from or without the generation of at least a part of the first stimuli.

10. Medical treatment device according to any one of claims 1 to 9, wherein the second stimuli, when being administrated to the patient's body during the second operation mode, suppress the pathologically synchronous activity of neurons.

11. Medical treatment device according to any one of claims 1 to 10, wherein in the first operating mode, a majority of the second stimuli, in particular more than 50% or 60%, and/or a majority of actuating periods within the sequence, in particular more than 50% or 60%, is coupled to the generation of at least one first stimuli.

12. Medical treatment device according to any one of claims 1 to 11, wherein the control unit is configured to, in the second operating mode, intermittently actuate the second stimulating device in a further sequence of actuating periods.

13. Medical treatment device according to any one of claims 1 to 12, wherein in the second operating mode, a majority of the second stimuli, in particular more than 50% or 60%, and/or a majority of actuating periods within the further sequence, in particular more than 50% or 60%, is de-coupled from the generation of the first stimuli.

14. Medical treatment device according to any one of claims 1 to 13, wherein the control unit is configured to switch from the first operating mode into the second operating mode by increasing, in particular successively increasing, the rate of second stimuli to be generated de-coupled from the generation of the first stimuli.

15. Medical treatment device according to any one of claims 1 to 14, wherein in the first and/or second operating mode, the generation of the second stimuli is coupled in time to the generation of the first stimuli such that in particular the second stimuli at least partially overlap the first stimuli, in particular by 10% to 100%.

16. Medical treatment device according to any one of claims 1 to 15, wherein n is an integer between one and a total number of different first stimuli to be generated by the first stimulating device.

17. Medical treatment device according to any one of claims 1 to 16, wherein the control unit is configured to, for at least one actuating period of the sequence, determine one first stimuli to be individually actuated during the at least one actuating period, and/or for at least one actuating period of the sequence, determine at least two first stimuli to be simultaneously actuated during the at least one actuating period, and/or determine at least three or more, for example three or four, first stimuli to be simultaneously actuated during the at least one actuating period.

18. Medical treatment device according to any one of claims 1 to 17, wherein the control unit, for each of the actuating periods of the sequence, is configured to stochastically and/or deterministically and/or combined stochastically-deterministically determine the number n of first stimuli to be generated during the respective actuating periods of the sequence.

19. Medical treatment device according to any one of claims 1 to 18, wherein the control unit, among the sequence of actuating periods, is configured to variedly select the determined number n of different first stimuli from the at least two first stimuli to be generated by the first stimulating device, wherein in particular the control unit is configure, for each of the actuating periods of the sequence, is configured to stochastically and/or deterministically and/or combined stochastically-deterministically determine the number n of different first stimuli from the at least two first stimuli to be generated by the first stimulating device.

20. Medical treatment device according to any one of claims 1 to 19, wherein the control unit is configured to stipulate resting periods between successive actuating periods of the sequence, wherein in particular the control unit is configured to stochastically and/or deterministically and/or combined stochastically-deterministically determine the duration of each of the resting periods.

21. Medical treatment device according to any one of claims 1 to 20, wherein to each one of the to first stimuli a different set of second stimuli is associate such that, during the first operating mode, each second stimuli to be paired to a respective first stimulus is selected from the set of second stimuli associated to the respective first stimulus, and wherein in particular the second stimuli differ among the sets of second stimuli.

22. Medical treatment device according to claim 21, wherein the control unit, for paring a second stimulus to a first stimulus during the first operating mode, is configured to stochastically and/or deterministically and/or combined stochastically-deterministically select at least one second stimulus from the set of second stimuli associated to the respective first stimulus.

23. Medical treatment device according to any one of claims 1 to 22, wherein the control unit is configured to, in the second operating mode, actuate the second stimulating device in a further sequence of actuating periods such that a number u of second stimuli to be generated simultaneously during respective actuating periods is variedly determined across the further sequence.

24. Medical treatment device according to claim 23, wherein u is an integer between one and a total number of different second stimuli to be generated by the second stimulating device.

25. Medical treatment device according to claim 23 or 24, wherein the control unit is configured to, for at least one actuating period of the further sequence, determine one second stimuli to be individually actuated during the at least one actuating period, and/or for at least one actuating period of the further sequence, determine at least two second stimuli to be simultaneously actuated during the at least one actuating period, and/or for at least one actuating period of the further sequence, determine at least three or more, for example three or four, second stimuli to be simultaneously actuated during the at least one actuating period.

26. Medical treatment device according to any one of claims 23 to 25, wherein the control unit, for each of the actuating periods of the further sequence, is configured to stochastically and/or deterministically and/or combined stochastically-deterministically determine the number u of second stimuli to be actuated during the respective actuating periods of the further sequence.

27. Medical treatment device according to any one of claims 23 to 26, wherein the control unit, among the further sequence of actuating periods, is configured to variedly select the determined number u of different second stimuli from the at least two second stimuli to be generated by the first stimulating device, wherein in particular the control unit, for each of the actuating periods of the sequence, is configured to stochastically and/or deterministically and/or combined stochastically-deterministically determine the number u of different second stimuli from the at least two second stimuli to be generated by the second stimulating device.

28. Medical treatment device according to any one of claims 23 to 27, wherein the control unit is configured to stipulate resting periods between successive actuating periods of the further sequence, wherein in particular the control unit is configured to stochastically and/or deterministically and/or combined stochastically-deterministically determine the duration of each of the resting periods.

29. Medical treatment device according to any one of claims 1 to 28, wherein the control unit is configured,
in a third operating mode, to actuate the second stimulating device in a third sequence of successive actuating periods such that a number u of first stimuli to be generated during the actuating periods is variedly determined across the sequence and to actuate the first stimulating device so as to generate the first stimuli to be paired to the generation of at least a part of the second stimuli, and
in a fourth operating mode, to actuate the second stimulating device so as to generate the first stimuli to be de-coupled from the generation of at least a part of the second stimuli.

30. Medical treatment device according to claim 29, wherein the control unit is configured to operate the first and the second stimulating device successively in the first operating mode, the second operating mode, the third operating mode and the fourth operating mode.

31. Medical treatment device according to any one of claims 1 to 30, further comprising a sensor unit for measuring stimulating effects on the neurons.

32. Medical treatment device according to claim 31, wherein the control unit is configured to operate the first and the second stimulating device in the respective operating modes in dependence on data measured by the sensor unit, wherein in particular the control unit is configured to switch the first and the second stimulating devices from the second operating mode into the first operating mode when a value indicative of the stimulating effect on the neurons measured by the sensor unit reaches a predefined threshold value.

33. Medical treatment device according to claim 31 or 32, wherein the control unit is configured to adapt the first and second stimuli generated by the first and second stimulating devices and/or the sequence of actuating periods in dependence on data measured by the sensor unit.

34. Medical treatment device according to any one of claims 31 to 33, wherein the control unit is further configured to adapt the further sequence of actuating periods in dependence on the data measured by the sensor unit.

35. Medical treatment device according to any one of claims 1 to 35, wherein the first and/or the second stimulating device is provided in the form of or comprised in a medical treatment glove, a medical treatment seat pad, a medical treatment sole, a medical treatment belly band, a medical treatment neck band, a medical treatment shoulder band, a medical treatment visual stimulation unit, and/or a medical treatment face mask.

36. Medical treatment device for stimulating neurons of a patient to suppress a pathologically synchronous activity of the neurons, the device comprises
a first non-invasive stimulating device for generating at least two different first stimuli to a patient's body and a second non-invasive stimulating device for generating at least two different second stimuli to the patient's body, wherein each of the first and the second stimuli are configured to, when being administrated to the patient's body, suppress the pathologically synchronous activity of neurons, and
a control unit for selectively and intermittently actuating the first and the second stimulating device, wherein the control unit is configured to:
in a first operating mode, actuate the first stimulating device in a first sequence of successive actuating periods, during which the first stimulating device generates at least one first stimuli, and to actuate the first stimulating device so as to generate the second stimuli to be paired to the generation of at least a part of the first stimuli, and
in a second operating mode, actuate the second stimulating device so as to generate the second stimuli to be de-coupled from the generation of at least a part of the first stimuli,
in a third operating mode, actuate the second stimulating device in a second sequence of successive actuating periods, during which the second stimulating device generates at least one second stimuli, and to actuate the first stimulating device so as to generate the first stimuli to be paired to the generation of at least a part of the second stimuli, and
in a fourth operating mode, actuate the first stimulating device so as to generate the first stimuli to be de-coupled from the generation of at least a part of the second stimuli.

37. Medical treatment device according to claim 37, wherein each of the first and the second stimuli is configured to, when being administrated to the patient's body, suppress the pathologically synchronous activity of neurons, in particular in at least one of a patient's brain or spinal cord.

38. Medical treatment device according to claim 37 or 38, wherein in the first operating mode, a majority of the second stimuli, i.e. more than 50 or 60%, is coupled to the generation of at least one first stimuli,
in the second operating mode, a majority of the second stimuli, i.e. more than 50% or 60%, is de-coupled from the generation of the first stimuli,
in the third operating mode, a majority of the first stimuli, i.e. more than 50% or 60%, is coupled to the generation of at least one second stimuli, and
in the fourth operating mode, a majority of the first stimuli, i.e. more than 50% or 60%, is de-coupled from the generation of the second stimuli.

39. Medical treatment device according to any one of claims 37 to 39, wherein the control unit is configured to operate the first and the second stimulating device successively in the first operating mode, the second operating mode, the third operating mode and the fourth operating mode.

40. Medical treatment method for stimulating neurons of a patient to suppress a pathologically synchronous activity of the neurons, the method comprising the steps of:
providing a first non-invasive stimulating device for generating at least two different first stimuli to a patient's body and a second non-invasive stimulating device for generating at least two different second stimuli to the patient's body, and
selectively and intermittently actuating the first and the second stimulating device successively in a first and a second operating mode, wherein
in the first operating mode, the first stimulating device is actuated in a sequence of successive actuating periods such that a number n of first stimuli to be generated simultaneously during the actuating periods is variedly determined across the sequence and the second stimulating device is actuated so as to generate the second stimuli to be paired to the generation of at least a part of the first stimuli, and wherein
in the second operating mode, the second stimulating device is actuated so as to generate the second stimuli to be de-coupled from the generation of at least a part of the first stimuli.
